(19) United States
(12) Patent Application Publication
(10) Pub. No.: US 2016/0123969 A1
(43) Pub. Date: May 5, 2016

(54) METHODS, MATERIALS, AND KITS FOR COVALENTLY ASSOCIATING MOLECULAR SPECIES WITH A SURFACE OF AN OBJECT

(71) Applicant: QUANTERIX CORPORATION, Lexington, MA (US)

(72) Inventors: David M. Rissin, Somerville, MA (US); David C. Duffy, Arlington, MA (US)

(73) Assignee: Quanterix Corporation, Lexington, MA (US)

(21) Appl. No.: 14/889,982

(22) PCT Filed: May 9, 2014
(86) PCT No.: PCT/US2014/037594

§ 371 Date: Nov. 9, 2015

Related U.S. Application Data
(60) Provisional application No. 61/821,389, filed on May 9, 2013.

(51) Int. Cl. G01N 33/543; G01N 33/551

(52) U.S. Cl. CPC G01N 33/54353; G01N 33/551

(57) ABSTRACT

Described herein are methods, materials, and kits for covalently associating molecular species with a surface of an object. The subject matter of the present invention involves, in some cases, interrelated products, alternative solutions to a particular problem, and/or a plurality of different uses of one or more systems and/or articles. In some aspects, methods are provided. In some embodiments, a method for covalently associating a molecular species with a surface comprises exposing an object with a surface comprising a plurality of functional groups to a first type of molecular species.

### RELATED APPLICATIONS

This application claims benefit under 35 U.S.C. §119(e) of U.S. provisional application Ser. No. 61/821,389 filed on May 9, 2013, the disclosure of which is incorporated herein by reference.

### FIELD OF THE INVENTION

Described herein are methods, materials, and kits for covalently associating molecular species with a surface of an object.

### BACKGROUND OF THE INVENTION

The ability to precisely measure multiple target analyte molecules simultaneously (e.g., proteins) is important in several fields, including clinical diagnostics, testing of blood banks, and the analysis of biochemical pathways. Multiplexed target analyte measurements provide richer information on the biological status of a sample compared to single target analyte measurements, while minimizing the use of sample volume and eliminating the need to run multiple assays. Various assays exist for the simultaneous detection of single molecules of multiple target analyte molecules (e.g., digital ELISA, see Rissin et al., Nat. Biotechnol. 2010, 28, 595-599, herein incorporated by reference). Certain digital ELISA assays involve capturing proteins on microscopic beads, labeling the target analytes with an enzyme, isolating the beads in arrays of small wells, and detecting bead-associated enzymatic activity using fluorescence imaging. In multiplexed digital ELISA, multiple subpopulations of beads each with a unique fluorescent signature and specific antibody can be incubated together in the same sample, and may be imaged simultaneously on the same array, e.g. within a microfluidic device. Spatial localization of individual beads in arrays enables the simultaneous determination of the single molecule signal associated with each bead subpopulation, enabling concentrations of multiple target analytes to be determined at very low concentrations. Various other multiplexed protein measurements have also been developed, many employing bead-based target analyte capture methods. Many of the assays rely on the ensemble signal from a large number of reporter molecules, which has limited their sensitivity—hundreds of labeled antibodies are required to reach instrument detection limits—and which therefore has limited their use in clinical diagnostics where analytical sensitivity is essential. This may be in part due to the interference of a signal of the bead with the signal employed for detecting the presence of the target analyte molecule. Accordingly, improved methods, materials, and kits are needed for multiplexed target analyte measurements, and more generally for other appropriate applications as well.

### SUMMARY OF THE INVENTION

Described herein are methods, materials, and kits for covalently associating molecular species with a surface of an object. The subject matter of the present invention involves, in some cases, interrelated products, alternative solutions to a particular problem, and/or a plurality of different uses of one or more systems and/or articles.

In some aspects, methods are provided. In some embodiments, a method for covalently associating a molecular species with a surface comprises exposing an object with a surface comprising a plurality of functional groups to a first type of molecular species, wherein at least some of the plurality of functional groups each covalently associate with the first type of molecular species and at least some of the plurality of functional groups do not associate with any of the first type of molecular species; deactivating the functional groups not associated with the first type of molecular species to form a plurality of deactivated functional groups; reactivating the plurality of deactivated functional groups to form a plurality of reactivated functional groups; and exposing the objects to a second type of molecular species, wherein at least some of the plurality of reactivated functional groups each covalently associate with a second type of molecular species.

In some aspects, materials are provided. In some embodiments, an activated material capable of being covalently functionalized with a first type of molecular species is provided comprising a plurality of functional groups associated with at least a portion of the surface of the activated material, wherein at least a portion of the functional groups are associated with the first type of molecular species; and at least a portion of the functional groups are not associated with the first type of molecular species but are instead deactivated and capable of being reactivated and of becoming covalently associated with a second type of molecular species.

### BRIEF DESCRIPTION OF THE DRAWINGS

Other aspects, embodiments, and features of the invention will become apparent from the following detailed description when considered in conjunction with the accompanying drawings. The accompanying figures are schematic and are not intended to be drawn to scale. For purposes of clarity, not every component is labeled in every figure, nor is every component of each embodiment of the invention shown where illustration is not necessary to allow those of ordinary skill in the art to understand the invention. All patent applications and patents mentioned in the text are incorporated by reference in their entirety. In case of conflict between the description contained in the present specification and a document incorporated by reference, the present specification, including definitions, will control.

FIG. 1 illustrates a non-limiting method for covalently associating molecular species with a surface, according to some embodiments;

FIG. 2 illustrates a non-limiting example of a method comprising the exposing, activating, and deactivating steps, according to some embodiments;

FIG. 3 illustrates a non-limiting example of an assay, according to some embodiments;

FIGS. 4A and 4B provide plots and graphs relating to examples of experiments used to determine cross-reactivity in multiplexed digital assays, according to some embodiments;

FIG. 5 shows representative images of an array from a multiplexed digital assay, according to some embodiments; and

FIGS. 6A and B shows plots of the average enzyme per bead against protein concentration for a non-limiting assay, according to some embodiments.

### DETAILED DESCRIPTION

Described herein are methods, materials, and kits for covalently associating molecular species with a surface of an object. The subject matter of the present invention involves, in some cases, interrelated products, alternative solutions to a particular problem, and/or a plurality of different uses of one or more systems and/or articles. It should be understood, that while much of the discussion below is directed to capture objects (e.g., beads), this is by way of example only, and other objects may be employed.

In some embodiments, methods for covalently associating a plurality of types of molecular species with a surface of an object are provided, as well as related materials and kits. Such methods may find use in a variety of applications, wherein the applications require use of a surface which is covalently associated with more than one type of molecular species. For example, the methods described herein may find use in the preparation of a plurality of capture objects (e.g., beads) for use in an assay, wherein the assay requires a plurality of types of capture objects, each type of capture object being uniquely identifiable and/or each type of capture object being adapted and arranged to associate with a particular target analyte molecule or particle. Such assays may be employed for characterizing, detecting, and/or quantifying a plurality of types of analyte molecules or particles in a sample.

In addition, the methods and kits described herein may provide advantages over previously described methods and kits comprising a plurality of types of molecular species covalently associated with the surface of an object. For example, in some embodiments, the methods or kits comprise a plurality of deactivated functional groups. The presence of and/or formation of deactivated functional groups may result in the objects being more stable under substantially similar conditions for greater periods of time as compared to objects comprising the functional groups which are not deactivated. The increased stability of the objects may allow for longer periods of storage and/or greater flexibility to further functionalize the objects. Alternatively or in addition, in some embodiments at least one of the types of molecular species comprises a reporter molecule (e.g., a dye). The methods and kits described herein may allow for association of a smaller amount of the reporter molecules as compared to previously described methods, which may be beneficial in embodiments wherein more than one type of detectable signal is to be interrogated and/or detected. For example, in an embodiment wherein the object is a bead and the bead is associated with a dye molecule and the presence or absence of the dye molecule is used to determine the presence of absence of the bead (e.g., using optical interrogation), lower concentrations of the dye molecule associated with the bead may be beneficial and provide for better results when the bead is employed in an assay wherein an analyte molecule is also detected using similar interrogation methods (e.g., an optical signal). That is, any interference from the signal of the bead with the signal associated with the presence of an analyte molecules may be reduced or eliminated as compared to conventional methods.

In some embodiments, a method for covalently associating a first type and a second type of molecular species with a surface is provided, which comprises exposing an object with a surface comprising a plurality of functional groups to a first type of molecular species. At least some of the plurality of functional groups each covalently associates with the first type of molecular species and at least some of the plurality of functional groups do not associate with any of the first type of molecular species. The functional groups not associated with the first type of molecular species may then be deactivated to form a plurality of deactivated functional groups. In some cases, the plurality of deactivated functional groups may be reactivated to form a plurality of reactivated functional groups. Upon exposure of the object to a second type of molecular species, at least some of the plurality of reactivated functional groups each covalently associates with a second type of molecular species.

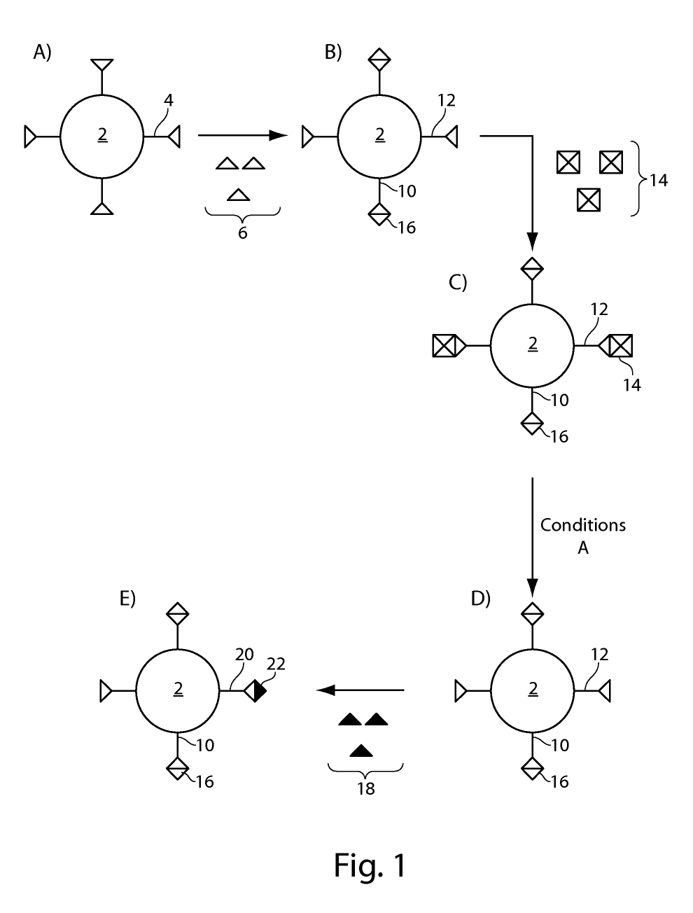

The above described method is depicted in FIG. 1. In FIG. 1, step A, object 2 (e.g., in this embodiment, a bead) is shown comprising a surface having a plurality of functional groups (e.g. 4) associated with the surface. Object 2 is exposed to a plurality of a first type of molecular species 6, and at least some of the plurality of functional groups each covalently associate with the first type of molecular species (e.g., functional group 10 is shown associated with a first type of molecular species 16) and least some of the plurality of functional groups do not associate with any of the first type of molecular species (e.g., functional group 12), as shown in FIG. 1, step B. The functional groups not associated with the first type of molecular species may then be deactivated to form a plurality of deactivated functional groups. In this embodiment, the plurality of functional groups not associated with any of the first type of molecular species are deactivated by exposing the object to a deactivating agent 14, wherein the deactivating agent reacts or associates with the functional groups to form a deactivated functional group (e.g., functional group 12 associates/reacts with deactivating species 14), as shown in FIG. 1, step C. The object formed in FIG. 1, step C comprising deactivated functional groups may then be exposed to conditions (e.g., conditions A), wherein the deactivated groups are reactivated to reform the functional groups, as shown in FIG. 1, step D. Finally, the object from FIG. 1, step D may be exposed to plurality of a second type of molecular species 18, and at least some of the reactivated groups associated with the second type of molecular species (e.g., reactivated functional group 20 is shown associated with second type of molecular species 22).

Those of ordinary skill in the art will be able to apply the methods described herein to covalently associate more than two types of molecular species with a surface. For example, in some embodiments, during the first exposing step, the object may be exposed to more than one type of molecular species (e.g., a first type and a third type of molecular species), wherein the object covalently associates with at least some of each of the types of molecular species and at least some of the functional groups do not associate with any molecular species. As another example, in addition or alternatively, following the deactivating/reactivating of the functional groups, the object may be exposed to more than the second type of molecular species (e.g., a second type and a fourth type of molecular species), wherein the object covalently associates with at least some of each of the second and fourth types of molecular species. As yet another example, in addition or alternatively, following exposing the object to a second type of molecular species, additional methods steps may be carried out, including exposing, activating, and/or deactivating steps, to associate a third type, or more, molecular species with the object. In some embodiments, two types, or three types, or four types, or five types, or six types, or more, of molecular species are associated with the object.

Other aspects of the methods will now be discussed in detail. It should be understood, that none, a portion of, or all of the following steps may be performed at least once during certain exemplary method formats described herein. Non-limiting examples of additional steps not described which may be performed include, but are not limited to, washing and/or exposure to additional reagents, exposure to additional types of molecular species, etc., as well as final deactivation/quenching step(s) to deactivate/quench any remaining functional groups which are not associated with a molecular species.

As described herein, in some embodiments, a method may comprise exposing an object with a surface comprising a plurality of functional groups to plurality of molecular species. The object may be exposed to the plurality of molecular species such that only a portion of the functional groups covalently associate with a molecular species (e.g., such that at least some of the plurality of functional groups each covalently associates with a molecular species and at least some of the plurality of functional groups do not associate with any molecular species). In some embodiments, this may be accomplished by limiting the amount of molecular species that the object is exposed to. For example, the concentration of the molecular species to which the object is exposed may be such that there is not enough molecular species to associate with each and every functional group. Alternatively and/or in addition, the time during which the object is exposed to the molecular species may be selected so that kinetically, there is not enough time for each and every functional group to associate with a molecular species. Those of ordinary skill in the art will be able to select conditions so that only a portion of the functional groups associate with a molecular species.

In some embodiments, the amount of the molecular species associated with an object may be optimized to limit any negative effects associated with too much or too little of the molecular species being associated with the object. For example, in embodiments wherein the molecular species is a reporter molecule, if too little of the reporter molecule is associated with the object, the object may not be detectable. Alternatively, if too much of the reporter molecule is associated with the object, one object may interfere with analyzing another object, or the reporter molecule may interfere with analyzing of another type of molecular species. In some embodiments, if various types of objects are to be employed in a single application (e.g., an assay comprising a plurality of types of objects), each type of object may be analyzed and the molecular species concentration optimized to minimize or eliminate any crossover readings or interference (e.g., different levels of the molecular species can be distinguished separately from the different types of beads; any other types of molecular species can be identified, etc.).

In some embodiments, the number of a type of molecular species associated with an object may be determined. In some embodiments, wherein the molecular species is a reporter molecule (e.g., dye), the method of determining the number of reporter molecules associated with an object may comprise determining a calibration curved for a particular reporter molecule. For example, in some cases, an unactivated object (e.g., wherein the functional groups are deactivated or no functional groups are present on the object such that the reporter molecule does not bind to the dye) may be mixed with a plurality of known concentrations of the reporter molecules. The calibration curve for a particular concentration of the reporter molecules may be generated by determining the average signal for a plurality of objects. To determine the amount of reporter molecules associated with an object (e.g., covalently associated), the object may be analyzed using the same or substantially similar techniques as those used to analyze the objects for generation of the calibration curve. The number of reporter molecules associated with the object may then be determined by comparing the signal for the object (or an average of a plurality of objects) to the calibration curve (e.g., via interpolation). See Example 2 for a non-limiting method of determining the number of reporter molecules per object for a plurality of objects, wherein the object is a bead.

In some embodiments, the number of molecular species (e.g., reporter molecules) per object is between about 100 and about 250,000, or between about 1000 and about 200,000, or between about 1000 and about 150,000, or between about 1000 and about 100,000, or between about 1000 and about 50,000.

In some embodiments, only a portion of the functional groups on the surface of an object are covalently associated with a type of molecular species. In some embodiments, the number of functional groups on the surface of an object can be estimated or determined, and based on the estimation or determination, the percentage of functional groups associated with a molecular species can be determined. Those of ordinary skill in the art will know of methods for estimating or determining the number of functional groups on the surface of an object. In some embodiments, the functional groups spacing may be estimated based on knowledge relating to self-assembled monolayers. For example, the spacing of the functional groups and the space occupied by each functional group may be estimated. Once these values are estimated, an estimated number of the functional groups on the surface may be calculated based upon to the total surface area comprising the functional groups. A range of the estimated number of functional groups may be determined by estimating a minimum and maximum spacing.

Any suitable method and/or chemistry may be employed for associating the types of molecules species with the surface. In some embodiments, each type of molecular species is associated with the surface via formation of a covalent bond. The method of the attachment of the molecular species to the surface depends of the type of molecular species and the nature of the surface and may be accomplished by a wide variety of suitable coupling chemistries/techniques. The type of functional groups present on the surface generally depends on the type of chemistry/method that is employed for covalently associating the types of molecular species to the surface of the object. Generally, the functional groups should be selected to be a group which is capable of covalently associating with each of the types of molecular species desired to be coupled, as well as being capable of being deactivated/reactivated.

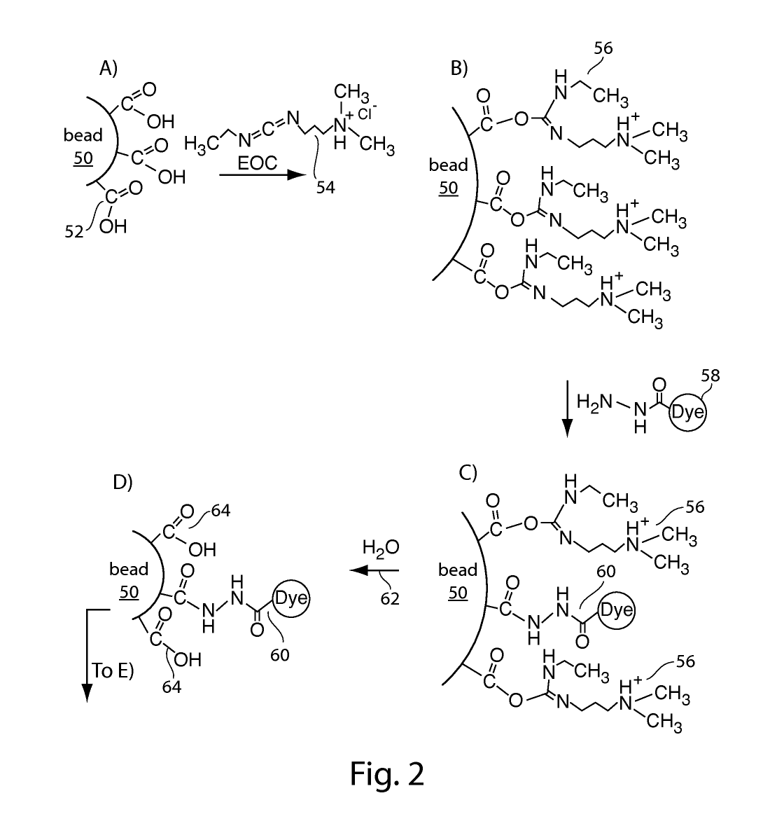

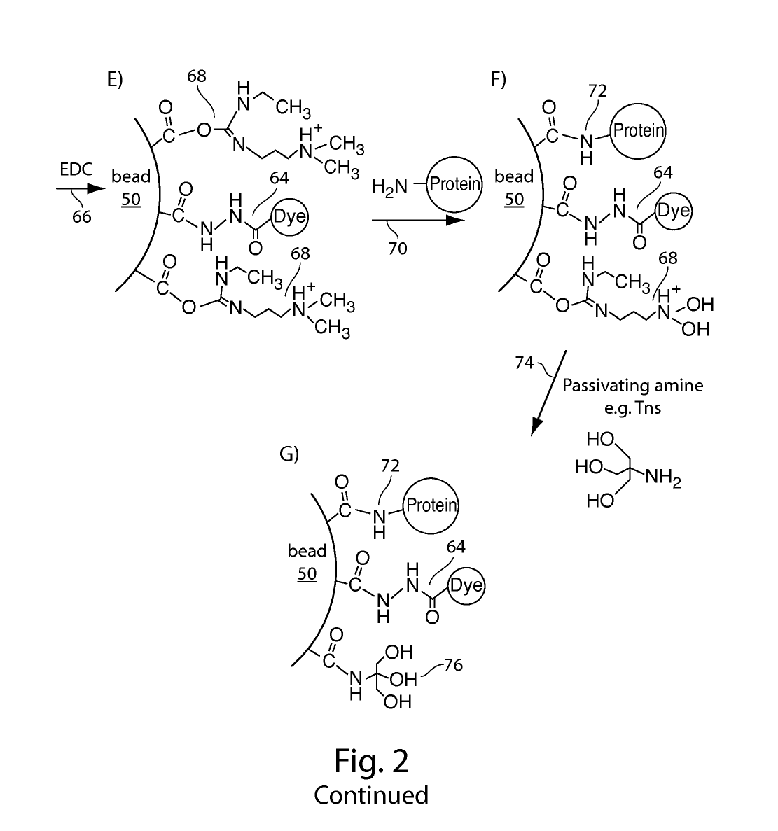

In certain embodiments, attachment of a molecular species to a surface may be accomplished via use of a chemical crosslinker. For example, a chemical crosslinker may be employed which comprises a group reactive with the molecular species and a group that is reactive with a group on the surface. The functional group may comprise a chemical crosslinker. As a specific example, the surface of an object may comprise a plurality of carboxylic acid groups. Next, the object is exposed to a chemical crosslinker, wherein one portion of the chemical crosslinker reacts with the carboxylic acid. Another portion of the crosslinker comprises a reactive component. The reactive component may react with a desired molecular species, facilitating covalent attachment of the molecular species to the surface of the object via the crosslinker. As described herein, upon association of a molecular species, a portion of the functional group (e.g. comprising the chemical crosslinker) may no longer be associated with the molecule following covalent reaction with the molecular species (e.g., see FIG. 2).

The exposing step may be carried out using techniques known to those of ordinary skill in the art. In some embodiments, the exposing step is conducted in a solution. For example, the surface may be exposed to a solution comprising the at least one type of molecular species and one or more solvents. The one or more solvents may be selected so that the at least one type of molecular species is soluble in the solvent(s). In some embodiments, additional reactants which aid in the covalent association between the functional group and the molecular species may be present in the solution. For example, in some embodiments, the solution may comprise an acid, a base, and/or a catalyst to assist in the reaction between the molecular species and the functional groups. Exemplary reactions and chemistries for forming a covalent association between a functional group and a molecular species are described herein. Similar conditions may be employed for any of the other method steps described herein, for example, the deactivating step, the reactivating step, and/or the association of a second type of molecular species.

Those of ordinary skill in the art will be aware of methods and techniques for exposing an object to a solution (e.g., comprising a type of molecular species, a deactivating agent, a reactivating agent, etc.). For example, the object may be added (e.g., as a solid, or in a solution/suspension) directly to the solution. As another example, the solution may be combined with a solution or suspension comprising the object and/or poured onto the surface of the object. In some instances, the solutions or suspensions may be agitated (e.g., stirred, shaken, etc.).

Examples of functional groups for attachment of the molecular species that may be useful include, but are not limited to, amino groups, carboxyl groups, epoxide groups, aldehyde groups, hydrazide groups, hydroxyl groups, hydrogen-reactive groups, maleimide groups, oxo groups, and thiol groups. In some embodiments, the functional group selected is capable of being deactivated and reactivated, as described in more detail herein. In some embodiments, upon association of a molecular species, a portion of the functional group may no longer be associated with the molecule following covalent reaction with the molecular species (e.g., see FIG. 2).

In some embodiments, the functional groups on the surface not associated with any molecular species may be deactivated, thereby forming a plurality of deactivated functional groups. The term deactivated is used herein to describe a functional group that has been deactivated so that its reactivity with itself, another portion of the object to which it is attached, or another substantially similar object to which it is exposed, is substantially decreased or eliminated, or to describe the process by which the deactivated functional group is formed. For example, in embodiments wherein the object comprises a plurality of functional groups, some of which are associated with a molecular species and some which are not, the functional group not associated with a molecular species could be reactive with the molecular species present on the object, or the molecular species present on a substantially similar object to which it is exposed (e.g., for embodiments where a plurality of objects are present). Deactivation of the functional groups reduces or eliminates the possibility of reaction between the functional group not associated with a molecular species and the molecular species present on the object, or the molecular species present on a substantially similar object to which is exposed. In addition, the deactivated functional group is capable of being reactivated. That is, the functional group may be reformed or otherwise restored or partially restored to reactivity upon activation of the deactivated group.

A functional group may be deactivated using any suitable method. In some embodiments, a functional group may be deactivated by exposing the functional group to a deactivating agent wherein the deactivating agent reacts or associates with the functional group to form a deactivated functional group. Alternatively, a functional group may be deactivated by exposing the functional group to conditions so that a portion of the functional group is detached. As yet another alternative, in some embodiments, a functional group may be deactivated by exposing the functional group to certain physical conditions. The deactivated functional group may be reactivated to form a reactivated functional group using any known method. The term reactivated is used herein to describe a deactivated functional group which has been returned to a more reactive state or substantially similar reactive state, or the process by which the deactivated functional group is returned to its original state. As will be understood by those of ordinary skill in the art, the method of reactivation will depend on the type of functional group and the method used for deactivation. Generally, the deactivation/reactivation conditions are selected so that the covalent association of any molecular species with the object is not affected. That is, the molecular species associated with the object remain associated with the object prior to, during, and/or following the deactivation and/or reactivation steps.

As a first non-limiting example of deactivation/reactivation, a functional group may comprise a chemical crosslinker associated with the surface via a surface moiety (e.g., a carboxylic acid moiety), and deactivation may comprises disassociation of the chemical crosslinker from the surface moiety. This may be accomplished by exposing the surface to a deactivating agent, wherein the deactivating agent interacts with functional group and causes dissociation of at least a portion of the functional group (e.g., the chemical crosslinker) from the surface. As a specific non-limiting example, the surface moiety may be a carboxylic acid residue and the chemical crosslinker may be 1-ethyl-3-[3-dimethylaminopropyl]carbodiimide hydrochloride (EDC), 1-cyclohexyl-3-(2-morpholinoethyl)carbodiimide (CMC), dicyclohexyl carbodiimide (DCC), or diisopropylcarbodiimide (DIC). The functional group comprising EDC associated with the carboxylic acid moiety may be deactivated by exposing the surface to conditions so that the EDC portion of the functional group dissociates from the carboxylic acid moiety (e.g., via hydrolization). To reactivate the functional group, the surface may be exposed to EDC and/or another reagent or combination thereof (for example, EDC and N-hydroxysuccinimide (NHS) or N-hydroxysulfosuccinimide (sulfo-NHS)), wherein EDC covalently associates with the carboxylic acid moiety to reform the functional group.

As a second non-limiting example, the functional group may comprise a reactive component, wherein upon exposure to a deactivating species, the reactive component covalently or otherwise associates with a portion of the deactivating agent. The deactivated functional group may be reactivated by exposure to conditions causing the portion of the deactivating agent associated with the deactivated functional group to dissociate from the functional group. As a specific non-limiting example, the functional group may be a thiol and the thiol may be deactivated by reaction with a deactivating species (e.g., reversible sulfhydryl blocking reagents, such as sodium tetrathionate and pyridyl disulfide containing compounds) wherein a disulfide is formed. The thiol may be reactivated by cleaving the disulfide to form the thiol using methods know in the art. For example, disulfide reducing agents such as dithiothreitol, 2-mercaptoethanol, 2-mercaptoethylamine, and tris(2-carboxylethyl)phosphine (TCEP), can be used to regenerate free thiol groups from disulfides. After washing away the reducing agent, the thiol group can then react to form irreversible bonds, e.g. thioesters (reaction with activated acyl groups), thioethers (reaction with activated akyl groups), and Michael addition products (reaction with maleimides).

As a third non-limiting example, the functional group may comprise a component which is deactivated by exposing the functional group to a set of physical conditions, for example, a change in temperature, a change in light exposure, etc. The deactivated functional group may be reactivated by reversal of the conditions. As a specific non-limiting example, the functional groups may comprise a photoreactive group (e.g., benzophenone), wherein the functional group is deactivated when not exposed to UV light, and is reactivated upon exposure to UV light.

Following deactivating/reactivation, the reactivated functional groups may then be used to covalently associate with a second type of molecular species. The covalent association of the second type of molecular species may be carried out using the same or similar techniques and methods as described herein for association of the first type of molecular species.

A non-limiting example of a specific method comprising exposing, activating, and deactivating steps is shown in FIG. 2. In FIG. 2, step A, a bead is provided, wherein the surface of bead 50 comprises a plurality of carboxylic acid groups (e.g., 52). The bead may be exposed to a crosslinker (e.g., EDC, 54), wherein a plurality of functional groups (e.g., 56) become associated with the bead surface, as shown in FIG. 2, step B. Upon exposure to a first type of molecular species, in this example, dye 58, a portion of the functional groups (e.g., 60) associate with the dye and a portion of the functional groups (e.g., 56) do not associate with any dye, as shown in FIG. 2, step C. The functional groups may then be deactivated. For example, as shown in FIG. 2, step D, the functional groups are deactivated in this example by hydrolysis (e.g., exposure to water 62), wherein the crosslinker agent is removed from the functional groups, and carboxylic acid groups (e.g., 64) are formed. Immediately or after any suitable period of time, the functional groups may be reactivated by exposure to the crosslinking agent once again (e.g., EDC 66), to form the reactivated functional groups (e.g., 68), and shown in FIG. 2, step E. The reactivated functional groups or a portion thereof may then be associated with a second type of molecular species. In this example, second type of molecular species 70 comprises a protein. As shown in FIG. 2, step F, bead 50 is covalently associated with dye 64 and protein 72, and at least some of the activated functional groups are not associated with either the dye or protein (e.g., 68). In some cases, any remaining activated functional groups may be deactivated, quenched, and/or associated with another type of molecular species. In this example, as shown in FIG. 2, step G, exposure of the bead to a passivating amine results in quenching of the activated functional group to form an inactivated functional group (e.g., 76).

During the method, one or more wash steps may be carried out using techniques known to those of ordinary skill in the art. A wash step may aid in the removal of any unbound molecules from the solution. A wash step may be performed using any suitable technique known to those of ordinary skill in the art, for example, by incubation of the objects with a wash solution followed by removal of the solution (e.g., in embodiments where small objects are employed such as beads, by centrifuging the solution comprising the objects and decanting off the liquid, or by using filtration techniques). In embodiments where the object is magnetic, the object may be isolated from the bulk solution with aid of a magnet.

Following covalent association of the desired types of molecular species, the object may be exposed to conditions such that any remaining non-reacted functional groups are deactivated. The deactivation may be carried out using one of the methods for deactivation described herein. Alternatively, following covalent association of the desired types of molecular species, the object may be exposed to conditions such that any remaining non-reacted functional groups are quenched (e.g., rendered inactive). That is, the inactivated functional groups cannot be readily reactivated upon exposure to reagents and/or physical conditions.

Any of a variety of suitable types of molecular species may be used in combination with the methods and materials described herein. Non-limiting examples of types of molecular species include reporter molecules (e.g., molecules which can be detected) or targeting entities (e.g., entities which target another specific molecule such as a target analyte molecule or particle, or a location). In some embodiments, the first type of molecular species is a reporter molecule and/or the second type of molecular species is a targeting entity (e.g., an antibody).

In some embodiments, an object is associated with more than one type of reporter molecule (e.g., two reporter molecules, three reporter molecules, etc.). The concentration of the types of reporter molecules may be varied so that different types of objects are distinguishable. For example, a first type of object may be associated with a first concentration of a first type of reporter molecules and a first concentration of a second type of reporter molecule and a second type of object may be associated with a second concentration of a first type of reporter molecules and a second concentration of a second type of reporter molecule. The first type of object and the second type of object may be distinguishable in embodiments wherein the first concentration of the first type of reporter molecule associated with the first type of object is different that the second concentration of the first type of reporter molecule associated with the second type of object and/or the first concentration of the second type of reporter molecule associated with the first type of object is different that the second concentration of the second type of reporter molecule associated with the second type of object. Alternatively or in addition, in some embodiments, an object is associated with more than one type of targeting entity (e.g., two targeting entities, three targeting entities, etc.).

As used herein, the term “reporter molecule(s)” refers to molecule(s) that give rise to a detectable signal (e.g., a fluorescent or chemiluminescent signal). Non-limiting examples of reporter molecules include fluorescent molecules, enzymes, dyes, and detectable particles (e.g., quantum dots). In some embodiments, the reporter molecule is a dye. Non-limiting examples of dyes include CF dyes (Biotium, Inc.), Alexa Fluor dyes (Invitrogen), DyLight dyes (Thermo Fisher), Cy dyes (GE Healthscience), IRDyes (Li-Cor Biosciences, Inc.), and HiLyte dyes (Anaspec, Inc.). In some embodiments, the dye is a hydrazide dye (e.g., Alexa Fluor® 488 hydrazide (AF-488), cyanine 5 hydrazide (cy5), and Hilyte Fluor® 750 hydrazide (HF-750)). In some embodiments, the excitation and/or emission wavelengths of a dye or reporter molecule are in the visible region (e.g., between about 400 nm and about 800 nm, or between 400 nm and about 750 nm). In some embodiments, the excitation and/or emission wavelengths of a dye or reporter molecule are in the UV region.

As used therein, the term “targeting entity” is any molecule or other chemical/biological entity that can be used to specifically attach, bind or otherwise capture a target molecule or particle (e.g., an analyte molecule), such that the target molecule/particle becomes immobilized with respect to the targeting entity or alternatively, targets a location (e.g., a location within a human). The immobilization, as described herein, may be caused by the association of an analyte molecule with the targeting entity. As used herein, “immobilized” means captured, attached, bound, or affixed so as to prevent dissociation or loss of the target molecule/particle, but does not require absolute immobility with respect to the targeting entity.

As will be appreciated by those in the art, the selection of the targeting entity will depend on the composition of what is being targeted (e.g., the target analyte molecule or particle). Targeting entities for a wide variety of target molecules are known or can be readily found or developed using known techniques. For example, when the target molecule is a protein, the targeting entity may comprise proteins, particularly antibodies or fragments thereof (e.g., antigen-binding fragments (Fabs), Fab′ fragments, pepsin fragments, F(ab′)₂ fragments, full-length polyclonal or monoclonal antibodies, antibody-like fragments, etc.), other proteins, such as receptor proteins, Protein A, Protein C, etc., or small molecules. In some cases, targeting entities for proteins comprise peptides. For example, when the target molecule is an enzyme, suitable targeting entities may include enzyme substrates and/or enzyme inhibitors. In some cases, when the target analyte is a phosphorylated species, the targeting entity may comprise a phosphate-binding agent. In addition, when the target molecule is a single-stranded nucleic acid, the targeting entity may be a complementary nucleic acid. Similarly, the target molecule may be a nucleic acid binding protein and the targeting entity may be a single-stranded or double-stranded nucleic acid; alternatively, the targeting entity may be a nucleic acid-binding protein when the target molecule is a single or double stranded nucleic acid. Also, for example, when the target molecule is a carbohydrate, potentially suitable targeting entity include, for example, antibodies, lectins, and selectins. As will be appreciated by those of ordinary skill in the art, any molecule that can specifically associate with a target molecule of interest may potentially be used as a targeting entity. For certain embodiments, suitable target analyte molecule/targeting entity pairs can include, but are not limited to, antibodies/antigens, antigens/antibodies, receptors/ligands, proteins/nucleic acid, nucleic acids/nucleic acids, enzymes/substrates and/or inhibitors, carbohydrates (including glycoproteins and glycolipids)/lectins and/or selectins, proteins/proteins, proteins/small molecules; small molecules/small molecules, etc.

Any suitable object may be used with the methods and materials described herein. The object may be fabricated from one or more suitable materials, for example, plastics or synthetic polymers (e.g., polyethylene, polypropylene, polystyrene, polyamide, polyurethane, phenolic polymers, or nitrocellulose etc.), naturally derived polymers (latex rubber, polysaccharides, polypeptides, etc.), composite materials, ceramics, silica or silica-based materials, carbon, metals or metal compounds (e.g., comprising gold, silver, steel, aluminum, copper, etc.), inorganic glasses, silica, and a variety of other suitable materials. Non-limiting examples of potentially suitable configurations include beads (e.g., magnetic beads), tubes (e.g., nanotubes), plates, disks, dipsticks, or the like.

In some embodiments, the object includes a binding surface having plurality of functional groups. The portion of the object which comprises a binding surface may be selected or configured based upon the physical shape/characteristics and properties of the objects (e.g., size, shape), and the format of the assay. In some embodiments, substantially all of the outer surfaces of the object comprise a plurality of functional groups. In some embodiments, association of a plurality of reporter molecules with an object allows for the object to be characterized as having an emission or absorption spectrum that can be exploited for detection so that location or other property of the object may be interrogated and/or determined.

According to one embodiment, each binding surface of an object comprises a plurality of functional groups. The plurality of functional groups, in some cases, may be distributed randomly on the binding surface like a “lawn.” Alternatively, the functional groups may be spatially segregated into distinct region(s) and distributed in any desired fashion or pattern.

In some embodiments, the object comprises a plurality of capture objects. The plurality of capture objects may be configured to be able to be spatially segregated from each other, that is, the capture objects may be provided in a form such that the capture objects are capable of being spatially separated into a plurality of locations. For example, the plurality of capture objects may comprise a plurality of beads (which can be of any shape, e.g., sphere-like, disks, rings, cube-like, etc.), a dispersion or suspension of particulates (e.g., a plurality of particles in suspension in a fluid), nanotubes, or the like. In some embodiments, the plurality of capture objects is insoluble or substantially insoluble in the solvent(s) or solution(s) utilized in an assay. In some cases, the capture objects are non-porous solids or substantially non-porous solids (e.g., essentially free of pores); however, in some cases, the plurality of capture objects may be porous or substantially porous, hollow, partially hollow, etc. The plurality of capture objects may be non-absorbent, substantially non-absorbent, substantially absorbent, or absorbent. In some cases, the capture objects may comprise a magnetic material, which as described herein, may facilitate certain aspect of an assay (e.g., washing step).

The object or plurality of capture objects may be of any suitable size or shape. Non-limiting examples of suitable shapes include spheres, cubes, ellipsoids, tubes, sheets, and the like. In certain embodiments, the average diameter (if substantially spherical) or average maximum cross-sectional dimension (for other shapes) of a capture object may be greater than about 0.1 um (micrometer), greater than about 1 um, greater than about 10 um, greater than about 100 um, greater than about 1 mm, or the like. In other embodiments, the average diameter of a capture object or the maximum dimension of a capture object in one dimension may be between about 0.1 um and about 100 um, between about 1 um and about 100 um, between about 10 um and about 100 um, between about 0.1 um and about 1 mm, between about 1 um and about 10 mm, between about 0.1 um and about 10 um, or the like. The “average diameter” or “average maximum cross-sectional dimension” of a plurality of capture objects, as used herein, is the arithmetic number average of the diameters/maximum cross-sectional dimensions of the capture objects. Those of ordinary skill in the art will be able to determine the average diameter/maximum cross-sectional dimension of a population of capture objects, for example, using laser light scattering, microscopy, sieve analysis, or other known techniques. For example, in some cases, a Coulter counter may be used to determine the average diameter of a plurality of beads.

In a particular embodiment, the objects comprise a plurality of beads. The beads may each comprise a plurality of functional groups associated with at least a portion of each bead. In some embodiments, the beads may be magnetic beads. The magnetic property of the beads may help in separating the beads from a solution and/or during washing step(s). Potentially suitable beads, including magnetic beads, are available from a number of commercial suppliers.

In some embodiments, activated materials are provided. The material may be capable of being covalently functionalized with a first type of molecular species. In some embodiments, the material comprises a plurality of functional groups associated with at least a portion of the surface of the activated material, wherein at least a portion of the functional groups are associated with the first type of molecular species and at least a portion of the functional groups are not associated with the first type of molecular species but are instead deactivated and capable of being reactivated rendering the functional group capable of becoming covalently associated with a second type of molecular species. For example, in some embodiments, a portion of the functional groups are associated with a protecting group that is capable of being removed to reexpose the functional group rendering the functional group capable of becoming covalently associated with a second type of molecular species. Other methods of deactivating functional groups are described herein. In some cases, the material comprises an object, as described herein. In some cases, the material comprises a plurality of beads.

In some embodiments, kits are provided. In some embodiments, the kit comprises a plurality of types of materials, wherein each type of material is uniquely identifiable. In some embodiments, each type of material may comprise a plurality of functional groups associated with at least a portion of the surface of the activated material, wherein at least a portion of the functional groups are associated (e.g., covalently associated) with the unique type or amount of a molecular species and at least a portion of the functional groups are not associated with the first type of molecular species but are instead deactivated and capable of being reactivated rendering the functional group capable of becoming covalently associated with another type of molecular species. Therefore each type of material is uniquely identifiable based on the unique type or amount of molecular species that is associated with the material. For example, each type of material may be covalently associated with a unique dye molecule and/or an unique amount of a dye molecule such that each type of material is uniquely identifiable based on the unique dye or unique amount of the dye. In some embodiments, a kit may comprise reagents and/or component necessary to reactivate the functional groups which are deactivated and/or the reagents and components necessary to associate a second type of molecular species with the material.

In some embodiments, the kit may optionally include instructions for use of the material. As used herein, “instructions” can define a component of instruction and/or promotion, and typically involve written instructions on or associated with packaging of the invention. Instructions also can include any oral or electronic instructions provided in any manner such that a user of the kit will clearly recognize that the instructions are to be associated with the kit. Additionally, the kit may include other components depending on the specific application, as described herein. As used herein, “promoted” includes all methods of doing business including methods of education, hospital and other clinical instruction, scientific inquiry, drug discovery or development, academic research, pharmaceutical industry activity including pharmaceutical sales, and any advertising or other promotional activity including written, oral and electronic communication of any form, associated with the invention.

In some embodiments, the objects may be detectable, e.g., by association of reporter molecule(s) with the object. In a specific embodiment, the objects are detectable optically. For example, the location of an object may be detected by identifying the optical signature of the object by a conventional optical train and optical detection system. Depending on the optical signature, and the operative wavelengths, optical filters designed for a particular wavelength may be employed for optical interrogation of the locations.

In some embodiments, the optical signal may be captured using a CCD camera. Other non-limiting examples of camera imaging types that can be used to capture images include charge injection devices (CIDs), complementary metal oxide semiconductors (CMOSs) devices, scientific CMOS (sCMOS) devices, and time delay integration (TDI) devices, as will be known to those of ordinary skill in the art. The camera may be obtained from a commercial source. CIDs are solid state, two dimensional multi pixel imaging devices similar to CCDs, but differ in how the image is captured and read. For examples of CIDs, see U.S. Pat. No. 3,521,244 and U.S. Pat. No. 4,016,550. CMOS devices are also two dimensional, solid state imaging devices but differ from standard CCD arrays in how the charge is collected and read out. The pixels are built into a semiconductor technology platform that manufactures CMOS transistors thus allowing a significant gain in signal from substantial readout electronics and significant correction electronics built onto the device. For example, see U.S. Pat. No. 5,883,830. CMOS devices comprise CMOS imaging technology with certain technological improvements that allows excellent sensitivity and dynamic range. TDI devices employ a CCD device that allows columns of pixels to be shifted into and adjacent column and allowed to continue gathering light. This type of device is typically used in such a manner that the shifting of the column of pixels is synchronous with the motion of the image being gathered such that a moving image can be integrated for a significant amount of time and is not blurred by the relative motion of the image on the camera. In some embodiments, a scanning mirror system coupled with a photodiode or photomultiplier tube (PMT) could be used to for imaging.

The objects described herein may find use in a variety of applications. In some embodiments, the objects may find use in applications comprising multiplexing. That is, wherein the application makes use of a plurality of types of objects, wherein each type of object is uniquely identifiable (e.g., via association with a unique type of reporter molecule or unique of reporter molecule amount) and uniquely targeted (e.g., via association of a unique target moiety, each unique targeting moiety being associated with a unique type or amount of reporter molecule). In some cases, the objects may comprise a plurality of beads, and the objects may be employed in the methods and systems described in U.S. patent application Ser. No. 12/731,130, entitled “Ultra-Sensitive Detection of Molecules or Particles using Beads or Other Capture Objects” by Duffy et al., filed Mar. 24, 2010, and issued as U.S. Pat. No. 8,236,574 on Aug. 7, 2012; U.S. patent application Ser. No. 12/731,136, entitled “Methods and Systems for Extending Dynamic Range in Assays for the Detection of Molecules or Particles” by Rissin et al., filed Mar. 24, 2010, and issued as U.S. Pat. No. 8,415,171 on Apr. 9, 2013; U.S. Patent Publication No. 2010/0075407 entitled “Ultra-Sensitive Detection of Molecules on Single Molecule Arrays” by Duffy et al., filed Sep. 23, 2008; U.S. Patent Publication No. 2010/0075439 entitled “Ultra-Sensitive Detection of Molecules by Capture-and-Release Using Reducing Agents Followed by Quantification” by Duffy et al., filed Sep. 23, 2008; U.S. Patent Publication No. 2010/0075355 entitled “Ultra-Sensitive Detection of Enzymes by Capture-and-Release Followed by Quantification” by Duffy et al., filed Sep. 23, 2008; U.S. Patent Publication No. 2011/0212462 entitled “Ultra-Sensitive Detection of Molecules Using Dual Detection Methods” by Duffy et al., filed Mar. 24, 2010; and U.S. Patent Publication No. 2011/0245097 entitled “Methods and Systems for Extending Dynamic Range in Assays for the Detection Of Molecules or Particles” by Rissin et al., filed Mar. 3, 2011, each herein incorporated by reference.

The following examples are included to demonstrate various features of the invention. Those of ordinary skill in the art should, in light of the present disclosure, will appreciate that many changes can be made in the specific embodiments which are disclosed while still obtaining a like or similar result without departing from the scope of the invention as defined by the appended claims. Accordingly, the following examples are intended only to illustrate certain features of the present invention, but do not necessarily exemplify the full scope of the invention

### Example 1

This example describes a method that enables the multiplexed detection of proteins based on counting single molecules. Paramagnetic beads were labeled with fluorescent dyes to create optically distinct subpopulations of beads, and antibodies to specific proteins were then immobilized to individual subpopulations. Mixtures of subpopulations of beads were then incubated with a sample, and specific proteins were captured on their specific beads; these proteins were then labeled with enzymes via immunocomplex formation. The beads were suspended in enzyme substrate, loaded into arrays of femtoliter wells—or Single Molecule Arrays (Simoa)—that were integrated into a microfluidic device (the Simoa disc). The wells were then sealed with oil, and imaged fluorescently to determine: a) the location and subpopulation identity of individual beads in the femtoliter wells, and b) the presence or absence of a single enzyme associated with each bead. The images were analyzed to determine the average enzyme per bead (AEB) for each bead subpopulation that provides a quantitative parameter for determining the concentration of each protein. This approach was used to simultaneously detect TNF-α, IL-6, IL-1α, and IL-1β in human plasma with single molecule resolution at subfemtomolar concentrations, i.e., 200- to 1000-fold more sensitive than current multiplexed immunoassays. The simultaneous, specific, and sensitive measurement of several proteins using multiplexed digital ELISA could enable more reliable diagnoses of disease.

### Methods and Materials

Materials.

2.7-μm-diam., carboxyl-functionalized paramagnetic beads were obtained from Agilent Technologies. 1-ethyl-3-(3-dimethylaminopropyl)carbodiimide hydrochloride (EDC) was purchased from Thermo Scientific. Tween 20, bovine serum albumin (BSA), and 2-(N-morpholino)ethanesulfonic acid (MES) were purchased from Sigma-Aldrich. Phosphate buffered saline (PBS) was from Amresco. Alexa Fluor 488 hydrazide was obtained from Life Technologies. Cyanine-5 (cy5) hydrazide was obtained from GE Healthcare. Hilyte 750 hydrazide was obtained from Anaspec. Antibodies and proteins were obtained from R&D Systems. Detection antibodies were biotinylated using standard methods as described previously (e.g., see Rissin, D. M., Fournier, D. R., Piech, T., Kan, C. W., Campbell, T. G., Song, L., Chang, L., Rivnak, A. J., Patel, P. P., Provuncher, G. K., Ferrell, E. P., Howes, S. C., Pink, B. A., Minnehan, K. A., Wilson, D. H., Duffy, D. C. Simultaneous detection of single molecules and singulated ensembles of molecules enables immunoassays with broad dynamic range, Anal. Chem. 2011, 83, 2279-2285, herein incorporated by reference). Streptavidin-β-galactosidase (SβG) was conjugated in the laboratory using protocols described previously (e.g., see Rissin, D. M., Fournier, D. R., Piech, T., Kan, C. W., Campbell, T. G., Song, L., Chang, L., Rivnak, A. J., Patel, P. P., Provuncher, G. K., Ferrell, E. P., Howes, S. C., Pink, B. A., Minnehan, K. A., Wilson, D. H., Duffy, D. C. Simultaneous detection of single molecules and singulated ensembles of molecules enables immunoassays with broad dynamic range, Anal. Chem. 2011, 83, 2279-2285, herein incorporated by reference.). Resorufin-β-D-galactopyranoside (RGP) was purchased from Life Technologies. Simoa discs—comprised of 24 arrays of femtoliter wells molded into cylic olefin polymer and bonded to a microfluidic manifold, as described previously (e.g., see Kan, C. W.; Rivnak, A. J.; Campbell, T. G.; Piech, T.; Rissin, D. M.; Mosl, M.; Peterca, A.; Niederberger, H.-P.; Minnehan, K. A.; Patel, P. P.; Ferrell, E. P.; Meyer, R. E.; Chang, L.; Wilson, D. H.; Fournier, D. R.; Duffy, D. C. Isolation and detection of single molecules on paramagnetic beads using sequential fluid flows in microfabricated polymer array assemblies, Lab Chip 2012, 12, 977-95, herein incorporated by reference.)—were obtained from Sony DADC. Fluorocarbon oil (Krytox®) was obtained from Dupont. De-indentified plasma samples from human donors were obtained from Bioreclamation.

Preparation of Populations of Fluorescently Labeled Capture Beads that Present Different Antibodies.

A stock solution of paramagnetic beads (2.3×10⁹ beads/mL) was vortexed for 5 s three times, and placed on rotary mixer for 15 min. 521 μL of bead solution (1.2×10⁹ beads) was pipetted into a 1.7-mL polypropylene tube. The beads were separated on a magnet and washed twice with 1 mL PBS+0.1% Tween 20, and twice with 1 mL PBS. The beads were resuspended in 1 mL of PBS and transferred into 15-mL polypropylene tube. 1 mg of the dye-hydrazide was dissolved in 100 μL PBS. A solution of 40 mg/mL EDC in MES buffer pH 6.2 was prepared. Sufficient PBS was first added to the tube to make the total reaction volume 10 mL, 2.4-213 μL of dye hydrazide solution was then added to the beads depending on the fluorescence level required, and 250 μL of 40 mg/mL EDC was added to the bead/dye suspension (see table below for exact volumes used). The tube was capped, inverted twice, vortexed intermittently for 10 s, and placed on a rotating mixer for 30 min. After separating the beads on a magnet, the beads were washed once with 5 mL PBS+0.1% Tween 20, resuspended in 1 mL of PBS+0.1% Tween 20, and transferred into a 1.7-mL polypropylene tube. After separating the beads on a magnet, the beads were washed 3 times with 1 mL of PBS+0.1% Tween 20, resuspended in 1 mL PBS+0.1% Tween 20, and placed on a rotating mixer for 1 h. After separating the beads on a magnet, the PBS+0.1% Tween 20 solution was removed, the beads were resuspended in 1 mL of 100 mM sodium bicarbonate buffer pH 9.3 added, and placed on a rotating mixer for 1 h. The beads were stored in 100 mM sodium bicarbonate buffer, pH 9.3 at 2-8° C. in an opaque container.

| | | | mL of | |
|---|---|---|---|---|
| | Encoding | | 40 mg/mL | mL |
| Dye type | level | | EDC | of 1x PBS |
| | | ul of 10 mg/mL | | |
| | | dye stock | | |
| Alexa Fluor 488 | 1 | 55.7 | 0.25 | 8.69 |
| Hilyte 750 | 1 | 213.0 | 0.25 | 8.54 |
| cy5 | 2 | 2.4 | 0.25 | 8.75 |
| | | uL of 1 mg/mL | | |
| | | dye stock | | |
| cy5 | 1 | 3.0 | 0.25 | 8.75 |

To conjugate an antibody to dye-encoded beads, 479 μL of encoded bead stock (1.2×10⁹ beads/mL=0.575×10⁹ beads) was pipetted into a 1.7-mL polypropylene tube. The beads were separated and washed 3 times with 0.01 M NaOH, followed by separation and washing 3 times with deionized water. The beads were separated and washed twice with PBS+0.1% Tween 20, followed by twice with 50 mM MES pH 6.2. A solution of 1 mg/mL capture antibody in 50 mM MES pH 6.2 was prepared. The beads were pelleted on a magnet, the buffer was aspirated, and 0.25 mL of 1 mg/mL capture antibody solution was added to the beads. The mixture of beads and solution of antibody was vortexed, and incubated on a rotation mixer for 30 min. A solution containing 0.1 mg/mL EDC in 50 mM MES pH 6.2 was prepared, and 0.25 mL of this solution was added to the bead/antibody solution. This mixture was vortexed and incubated on the rotation mixer for 30 min, and the beads were separated and washed 3 times with PBS. 1 mL of 1% BSA in PBS was added to the beads and incubated for 60 min on the rotation mixer. The beads were washed twice with PBS, and stored at 2-8° C. in a buffer containing 500 mM Tris+1% BSA+0.1% Tween 20+0.15% Proclin 300 antimicrobial.

Capture of Multiple Proteins on Subpopulations of Magnetic Beads and Formation of Enzyme-Labeled Immunocomplexes.

500,000 beads of each of the four subpopulations presenting antibodies to the four proteins were mixed, pelleted, and the supernatant was aspirated. Test solutions (100 μL) were added to the mixture of the 2,000,000 magnetic beads and incubated for 2 h at 23° C. The beads were then separated and washed three times in 5×PBS and 0.1% Tween-20. The beads were resuspended and incubated with solutions containing mixtures of biotinylated detection antibodies (anti-TNF-α at 0.1 μg/mL; anti-IL-6 at 0.15 μg/mL; anti-IL-1α at 0.1 μg/mL; and anti-IL-1β at 0.3 μg/mL) for 60 min at 23° C. The beads were then separated and washed three times in 5×PBS and 0.1% Tween-20. The beads were incubated with solutions containing SβG (35 pM) for 30 min at 23° C., separated, washed seven times in 5×PBS and 0.1% Tween-20, and washed once in PBS. 1 million beads were then resuspended in 120 μL of 100 μM RGP in PBS, and 15 μL of this bead solution was loaded into a Simoa disc. The bead manipulation steps were performed on a Tecan EVO liquid handling system.

Loading and Sealing of Beads in Femtoliter-Volume Well Arrays.

A Simoa disc composed of 24 3×4 mm arrays of ˜216,000 femtoliter wells and individually addressable microfluidic manifolds was placed on the platen of a customized system developed by Stratec Biomedical for the load, seal, and imaging of the arrays. The design of this microfluidic device and related details of its operation are described (e.g., see Kan, C. W.; Rivnak, A. J.; Campbell, T. G.; Piech, T.; Rissin, D. M.; Mosl, M.; Peterca, A.; Niederberger, H.-P.; Minnehan, K. A.; Patel, P. P.; Ferrell, E. P.; Meyer, R. E.; Chang, L.; Wilson, D. H.; Fournier, D. R.; Duffy, D. C. Isolation and detection of single molecules on paramagnetic beads using sequential fluid flows in microfabricated polymer array assemblies, Lab Chip 2012, 12, 977-95, herein incorporated by reference.). For each sample analyzed, 15 μL of the solution containing the mixture of bead subpopulations and RGP was pipetted manually into the inlet port of the disc. Vacuum pressure was then applied to the outlet port and drew the bead solution over the arrays of femtoliter wells. The beads were allowed to settle via gravity onto the wells of the array for 2 min. After the beads had settled, 50 μL of fluorocarbon oil was automatically dispensed by the system in the inlet port, and vacuum was simultaneously applied to the outlet port to pull the oil over the array. The oil pushed the aqueous solution and beads that were not in wells off the array surface, and formed a liquid-tight seal over the wells containing beads and enzyme substrate as described previously.

Imaging of Single Molecules and Fluorescent Beads in Femtoliter-Volume Well Arrays.

Once the wells were sealed, a customized optical arrangement in the load, seal, and image system performed the imaging steps necessary for identifying which bead types were in which well, and whether enzyme activity was associated with the beads. The fluorescence-based optical system (developed by Stratec Biomedical) was composed of: a white light illumination source; a custom, 12-element, infinite conjugate lens system capable of wide-field-of-view imaging of 3×4 mm; a CCD camera (Allied Vision, Prosilica GT3300 8 Mp). The imaging process took 45 s in total for each array, and was composed of the following sequential steps. First, a “dark field” image of the array was acquired by using the 622 nm/615 nm excitation/emission filters (exposure time=0.3 ms). Second, an image at 574 nm/615 nm excitation/emission (exposure time=3 s) was acquired; this image is the t=0 image (F1) of the single molecule resorufin signal. Third, an image at excitation/emission of 740 nm/800 nm (exposure time=9 s) was acquired to identify beads labelled with the HF-750 dye. Fourth, an image at excitation/emission of 680 nm/720 nm (exposure time=3 s) was acquired; this image was not used in this work. Fifth, an image at excitation/emission of 622 nm/667 nm (exposure time=3 s) was acquired to identify beads labelled with the cy5 dye. Sixth, an image at 574 nm/615 nm excitation/emission (exposure time=3 s) was acquired 30 s after the image F1; this image is the t=30 s image (F2) of the single molecule resorufin signal. Finally, an image at excitation/emission of 490 nm/530 nm (exposure time=2 s) was acquired to identify beads labelled with the AF-488 dye. Images were saved as a single IPL file.

Analysis of Images.

A custom image analysis software program was used to determine the enzyme activity associated with each bead within each subpopulation from the captured images. An algorithm first identified and removed occlusions (such as bubbles and dust) from the images. A masking method was then applied to the dark field image to define the locations and boundaries of the wells. The resulting well mask was then applied to each of the fluorescence images to determine the presence of beads and enzymes within the wells. For the bead fluorescence images, histograms of fluorescence intensity were generated for the well population. Peaks in the histograms were identified automatically and used to determine the populations of empty wells (low fluorescence), and populations of single beads at a particular fluorescence level for each fluorescence wavelength. The well mask was also applied to the difference between the second and first frame at the resorufin wavelengths, i.e., F2−F1. Wells that had been classified as containing a single bead from a particular bead subpopulation were classified as: a) associated with enzyme activity (“on” or active), if the fluorescence from resorufin within that well increased beyond a known threshold, or; b) not associated with enzyme activity (“off” or inactive), if the fluorescence from resorufin within that well did not increase beyond a known threshold. For each “on” bead the intensity increase was determined. For each bead subpopulation, the fraction of “on” beads (fₒₙ) was determined. In the digital range (fₒₙ<0.7), fₒₙ was converted to average number of enzymes per bead (AEB) using the Poisson distribution equation as described previously (e.g., see Rissin, D. M., Fournier, D. R., Piech, T., Kan, C. W., Campbell, T. G., Song, L., Chang, L., Rivnak, A. J., Patel, P. P., Provuncher, G. K., Ferrell, E. P., Howes, S. C., Pink, B. A., Minnehan, K. A., Wilson, D. H., Duffy, D. C. Simultaneous detection of single molecules and singulated ensembles of molecules enables immunoassays with broad dynamic range, Anal. Chem. 2011, 83, 2279-2285, herein incorporated by reference). In the analog range (fₒₙ>0.7), AEB was determined from the average increase in fluorescence of all the beads in an array as described previously (e.g., see Rissin, D. M., Fournier, D. R., Piech, T., Kan, C. W., Campbell, T. G., Song, L., Chang, L., Rivnak, A. J., Patel, P. P., Provuncher, G. K., Ferrell, E. P., Howes, S. C., Pink, B. A., Minnehan, K. A., Wilson, D. H., Duffy, D. C. Simultaneous detection of single molecules and singulated ensembles of molecules enables immunoassays with broad dynamic range, Anal. Chem. 2011, 83, 2279-2285, herein incorporated by reference). During classification of beaded wells and determination of enzyme activity, the fluorescence and location of wells were corrected for the following: optical blurring and scattering, background non-uniformity, intra-well bead settling locations, wavelength-dependent refraction differences in the lens assembly, and bleed of fluorescence of dyes outside their dominant wavelengths.

### Results and Discussion

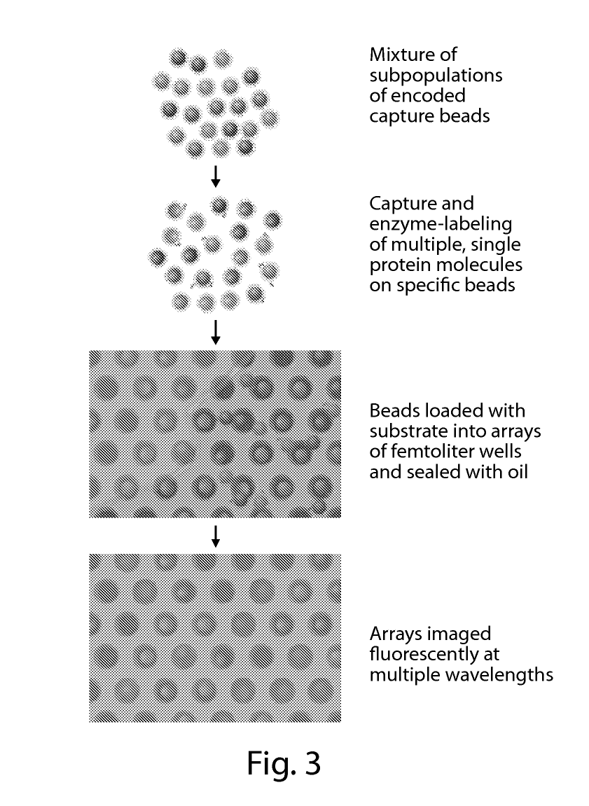

The measurement of single proteins using digital ELISA has been described in detail previously (e.g., see Rissin, D. M.; Kan, C. W.; Campbell, T. G.; Howes, S. C.; Fournier, D. R.; Song, L.; Piech, T.; Patel, P. P.; Chang, L.; Rivnak, A. J.; Ferrell, E. P.; Randall, J. D.; Provuncher, G. K.; Walt, D. R.; Duffy, D. C. Single-molecule enzyme-linked immunosorbent assay detects serum proteins at subfemtomolar concentrations, Nat. Biotechnol. 2010, 28, 595-599 and Rissin, D. M., Fournier, D. R., Piech, T., Kan, C. W., Campbell, T. G., Song, L., Chang, L., Rivnak, A. J., Patel, P. P., Provuncher, G. K., Ferrell, E. P., Howes, S. C., Pink, B. A., Minnehan, K. A., Wilson, D. H., Duffy, D. C. Simultaneous detection of single molecules and singulated ensembles of molecules enables immunoassays with broad dynamic range, Anal. Chem. 2011, 83, 2279-2285, herein incorporated by reference). In multiplexed digital ELISA (FIG. 3), subpopulations of microscopic beads each with their own unique fluorescent signature were created. Capture antibodies that bind a specific target protein were then immobilized on each subpopulation of beads. The subpopulations of beads were combined and incubated with a sample. An immunoassay sandwich was then formed by capture of the specific proteins on the corresponding subpopulations of beads, followed by sequential labeling of these proteins using a mixture of corresponding specific, biotinylated detection antibodies, and a common enzyme reporter molecule, streptavidin-β-galactosidase (SβG). The beads were suspended in a fluorogenic substrate of SβG, and loaded into a microfluidic device (the “Simoa disc” (e.g., see Kan, C. W.; Rivnak, A. J.; Campbell, T. G.; Piech, T.; Rissin, D. M.; Mosl, M.; Peterca, A.; Niederberger, H.-P.; Minnehan, K. A.; Patel, P. P.; Ferrell, E. P.; Meyer, R. E.; Chang, L.; Wilson, D. H.; Fournier, D. R.; Duffy, D. C. Isolation and detection of single molecules on paramagnetic beads using sequential fluid flows in microfabricated polymer array assemblies, Lab Chip 2012, 12, 977-95, herein incorporated by reference)) containing a 3×4 mm array of ˜216,000 femtoliter-sized microwells micromolded in cyclic olefin polymer. The microfluidic design of the Simoa disc has been described previously (e.g., see Kan, C. W.; Rivnak, A. J.; Campbell, T. G.; Piech, T.; Rissin, D. M.; Mosl, M.; Peterca, A.; Niederberger, H.-P.; Minnehan, K. A.; Patel, P. P.; Ferrell, E. P.; Meyer, R. E.; Chang, L.; Wilson, D. H.; Fournier, D. R.; Duffy, D. C. Isolation and detection of single molecules on paramagnetic beads using sequential fluid flows in microfabricated polymer array assemblies, Lab Chip 2012, 12, 977-95, herein incorporated by reference); the use of a micromolded microfluidic device provided the large numbers of wells, low fluorescence, and simple fluidic sealing to enable multiplexed Simoa. The wells of the array were sealed using fluorocarbon oil to prevent diffusion of the fluorescent product out of the wells (e.g., see Kan, C. W.; Rivnak, A. J.; Campbell, T. G.; Piech, T.; Rissin, D. M.; Mosl, M.; Peterca, A.; Niederberger, H.-P.; Minnehan, K. A.; Patel, P. P.; Ferrell, E. P.; Meyer, R. E.; Chang, L.; Wilson, D. H.; Fournier, D. R.; Duffy, D. C. Isolation and detection of single molecules on paramagnetic beads using sequential fluid flows in microfabricated polymer array assemblies, Lab Chip 2012, 12, 977-95, herein incorporated by reference). A bead associated with a single enzyme label generates a locally high concentration of fluorescent product in the sealed 50-fL well, making it possible to image single molecules. After sealing, the array was fluorescently imaged at the excitation/emission wavelengths of the enzyme product and the different dyes used to label the subpopulations of beads.

A customized Simoa imaging system was used to image ˜200,000 wells in single exposures at submicron resolution at five emission wavelengths. Based on these images, it was possible to determine the location in the femtoliter well arrays of thousands of beads from each subpopulation (“decoding”), and whether or not these beads were associated with enzyme activity. At femtomolar concentrations of proteins, the number of target molecules in a sample is smaller than the number of beads in a subpopulation, so the key measurement is the fraction of active, enzyme-associated (“on”) beads or fₒₙ (e.g., see Rissin, D. M., Fournier, D. R., Piech, T., Kan, C. W., Campbell, T. G., Song, L., Chang, L., Rivnak, A. J., Patel, P. P., Provuncher, G. K., Ferrell, E. P., Howes, S. C., Pink, B. A., Minnehan, K. A., Wilson, D. H., Duffy, D. C. Simultaneous detection of single molecules and singulated ensembles of molecules enables immunoassays with broad dynamic range, Anal. Chem. 2011, 83, 2279-2285, herein incorporated by reference). In multiplex digital ELISA, the combination of spatial separation of beads and bead encoding was used to determine fₒₙ independently for each protein, and then convert that to average enzymes per bead (AEB) via Poisson statistics (e.g., see Rissin, D. M., Fournier, D. R., Piech, T., Kan, C. W., Campbell, T. G., Song, L., Chang, L., Rivnak, A. J., Patel, P. P., Provuncher, G. K., Ferrell, E. P., Howes, S. C., Pink, B. A., Minnehan, K. A., Wilson, D. H., Duffy, D. C. Simultaneous detection of single molecules and singulated ensembles of molecules enables immunoassays with broad dynamic range, Anal. Chem. 2011, 83, 2279-2285, herein incorporated by reference). At values of fₒₙ less than about 0.7, Poisson statistics indicate that the majority of active beads are associated with a single enzyme, giving multiplexed digital ELISA its single molecule sensitivity. At higher concentrations, where essentially every bead is associated with at least one enzyme, the AEB from the average fluorescence intensity of all of the beads imaged for each subpopulation was determined (e.g., see Rissin, D. M., Fournier, D. R., Piech, T., Kan, C. W., Campbell, T. G., Song, L., Chang, L., Rivnak, A. J., Patel, P. P., Provuncher, G. K., Ferrell, E. P., Howes, S. C., Pink, B. A., Minnehan, K. A., Wilson, D. H., Duffy, D. C. Simultaneous detection of single molecules and singulated ensembles of molecules enables immunoassays with broad dynamic range, Anal. Chem. 2011, 83, 2279-2285, herein incorporated by reference). To determine the concentrations of multiple proteins in an unknown sample, calibration curves of AEB against known concentrations of protein mixtures were generated, and then interpolated concentrations from measured AEB values of unknowns.

A challenge for multiplexing digital ELISA was the potential for interference of the single enzyme signal by bead fluorescence. Single enzymes are detected by measuring fluorescence emitted from resorufin at 615±22 nm, and it was imperative that fluorescence from beads at this wavelength was low because the amount of resorufin produced from a single enzyme molecule is relatively small. Methods for fluorescently labeling beads via encapsulation or attachment of specific dye molecules that enable their encoding and decoding are well established. The amount of fluorescent dye encapsulated in commercial beads, however, is extremely high, resulting in unacceptably high fluorescent signal in the resorufin emission band, so that single molecules could not be detected. Therefore, a method was developed to label beads with multiple levels of individual dyes without interfering with detection of single enzymes. To encode beads with specific dyes and intensity levels, dye molecules were covalently attached to carboxyl beads, the unreacted activated carboxyl groups were hydrolyzed, and then capture antibodies were covalently attached via regenerated carboxyl groups. This approach had no adverse effect on assay performance when compared to beads coupled with antibody only. Alexa Fluor® 488 hydrazide (AF-488), cyanine 5 hydrazide (cy5), and Hilyte Fluor® 750 hydrazide (HF-750) dyes were used to encode bead types for multiplexed digital ELISA. By precisely controlling the ratio of encoding dye molecules to beads, discrete encoding levels for each dye were prepared, yielding subpopulations of beads that can be distinguished on the Simoa imager. Histograms of the fluorescence of four bead subpopulations were obtained: single levels of AF-488 and HF-750, and two levels of cy5. Automated software was used to identify bead subpopulations from these histograms as described in the Methods section. The fluorescence from these four bead populations did not significantly change the signals in the resorufin channel, allowing the detection of single enzyme molecules (see Table 1).

TABLE 1
Effect of fluorescence of unmodified and encoded beads on
the channel used to detect fluorescence (resorufin) from the
reaction of single enzymes.

| | Average fluorescence in resorufin |
|---|---|
| | detection channel (574 nm/615 nm |
| Bead type | ex/em) |
| Unmodified, non-encoded beads | 408 ± 14 |
| AF-488 fluorescent beads | 390 ± 9 |
| cy5 fluorescent beads (low) | 401 ± 12 |
| cy5 fluorescent beads (high) | 408 ± 11 |
| HF-750 fluorescent beads | 420 ± 12 |

Another challenge was to make sure that interactions between the bead subpopulations did not result in false positive Simoa signals. A false positive is defined as counting of a single enzyme associated with the bead intended to capture a specific protein that does not originate from capture and labeling of that particular protein. Digital ELISA measurements of single proteins can have false positive signals from the interaction of detection antibodies and enzyme with the capture beads in the absence of target protein molecules. In single-plex these false positives result in a consistent background that provides a useful noise floor for Simoa. In multiplexed digital ELISA, false positives may be more problematic because any interaction between bead subpopulations of a high abundance protein and a low abundance protein may increase the number of positive beads counted for the latter. Two sources of false positives were investigated: optical cross-talk and cross-reactivity of reagents.

Optical cross-talk occurs when signal from one well optically scatters into its neighboring wells. Optical scatter of fluorescence from resorufin produced by many enzyme labels on a bead into a neighboring well containing a bead with no enzyme label could result in the “off” bead actually appear as if it is associated with an enzyme, and be incorrectly identified as “on”. As a result, the AEB value for that protein may be falsely elevated. Analysis of images of high AEB bead subpopulations (and bright encoded beads) indicated that crosstalk in this example, was on the order of ≦1-2%, meaning that the likelihood of false positive signals from a low abundance analyte (AEB≈0.01) can increase if its beads are adjacent to those of an analyte at much higher concentrations (AEB>1). To reduce the impact of optical scatter, a computational method was developed for its active correction of each array based on analysis of the average scatter of encoded beads that have no neighboring beads. First, a “crosstalk-free” baseline was determined from the mean of the resorufin signal growth of non-beaded wells having only non-beaded neighbors. Second, the fraction of fluorescence crosstalk was determined from the average signal growth above baseline of non-beaded wells adjacent to only one positive, beaded neighboring well in each of the 6 nearest neighbor directions. Third, the signals of each beaded well was corrected by subtracting the weighted, directional mean of crosstalk based on the intensity of each of the beaded nearest neighbors. This correction allowed for the reduction of false positive calls when both high and low abundance proteins were present (Table 2).

TABLE 2
AEB values of 4 bead types in a 4-plex measured in samples spiked
with IL-6 before and after software correction of crosstalk.
Crosstalk was observed at 100 pg/mL IL-6 in all three non-IL-6
bead types, and these false positive signals are greatly reduced
by correction without affecting the IL-6 bead data.

| Beads | | | | | | | |
|---|---|---|---|---|---|---|---|
| mea- | [IL-6] | Before crosstalk correction | | | After crosstalk correction | | |
| sured | pg/mL | AEB | s.d. | CV | AEB | s.d. | CV |
| IL-6 | 0 | 0.012 | 0.001 | 8.0% | 0.012 | 0.001 | 8.3% |
| beads | 1 | 0.103 | 0.007 | 6.4% | 0.103 | 0.007 | 6.7% |
| | 10 | 0.921 | 0.021 | 2.2% | 0.922 | 0.021 | 2.2% |
| | 100 | 6.187 | 0.098 | 1.6% | 6.188 | 0.093 | 1.5% |
| TNF-α | 0 | 0.019 | 0.001 | 7.5% | 0.019 | 0.001 | 7.5% |
| beads | 1 | 0.020 | 0.001 | 5.1% | 0.021 | 0.001 | 5.5% |
| | 10 | 0.021 | 0.000 | 0.9% | 0.021 | 0.000 | 1.5% |
| | 100 | 0.060 | 0.001 | 1.9% | 0.031 | 0.003 | 10.0% |
| IL-1β | 0 | 0.021 | 0.001 | 6.0% | 0.021 | 0.001 | 6.4% |
| beads | 1 | 0.023 | 0.001 | 5.2% | 0.023 | 0.001 | 6.1% |
| | 10 | 0.023 | 0.004 | 15.7% | 0.023 | 0.004 | 15.6% |
| | 100 | 0.060 | 0.002 | 3.9% | 0.031 | 0.000 | 0.1% |
| IL-1α | 0 | 0.018 | 0.003 | 16.1% | 0.018 | 0.003 | 17.1% |
| beads | 1 | 0.023 | 0.003 | 12.2% | 0.023 | 0.003 | 13.0% |
| | 10 | 0.023 | 0.001 | 3.1% | 0.023 | 0.001 | 3.7% |
| | 100 | 0.069 | 0.001 | 0.9% | 0.033 | 0.001 | 1.5% |

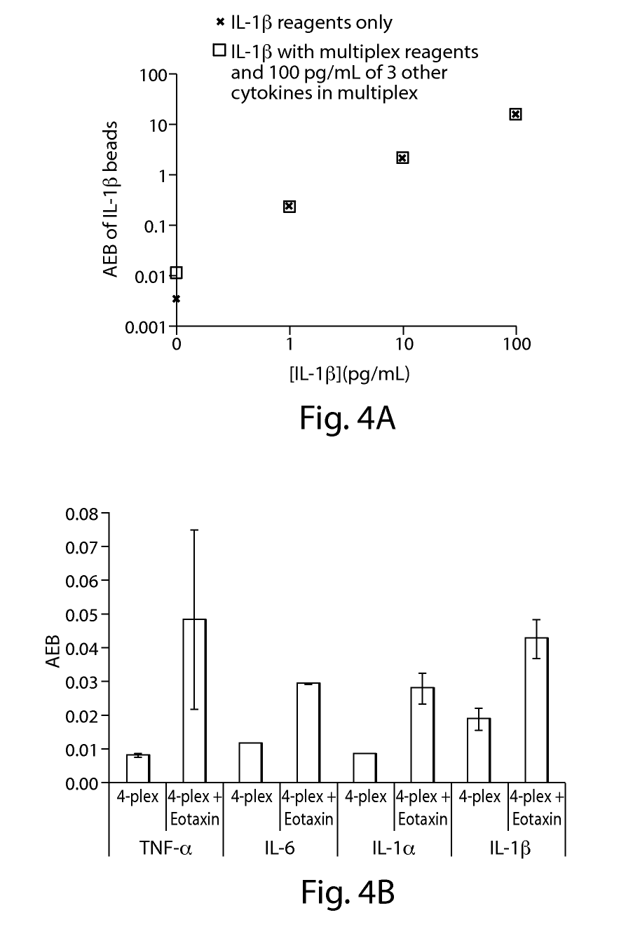

Cross-reactivity of immunological reagents is a source of false positive signals in immunoassays in general. If the antibodies used to detect protein “A” also bound another protein “B” in the multiplex with sufficient affinity that protein “B” was captured and measured on protein “A” beads at similar concentrations, then the specificity and dynamic range of the multiplex may be poor, limiting its usefulness. False positive signals from cross-reactivity between the reagents used to detect each cytokine in the multiplex were minimized. For each new protein added to a multiplex, “drop out” experiments were performed to demonstrate that the protein or antibody reagents did not cause false positive signals in the single-plex assay of the new protein or in the existing multiplex assay, as described herein and in FIG. 4 and Table A.

In FIG. 4: Examples of experiments to determine cross-reactivity in multiplexed digital ELISA. A) IL-1β was being added to an existing 3-plex of TNF-α, IL-6, and GM-CSF. IL-1β beads were run in conventional singleplex mode (crosses), and also with 100 pg/mL each of TNF-α, IL-6, and GM-CSF, and a mixture of the biotinylated detection antibodies for these 3 cytokines added to the assay (squares). The 3-fold increase in background signals for IL-1β beads was expected from the use of four-fold higher concentration of detection antibodies, but no further increase was observed from the presence of 100 pg/mL of 3 other antigens, so cross-reactivity was acceptable. B) Eotaxin was being added to an existing 4-plex of TNF-α, IL-6, IL-1α, and IL-1β. The 4-plex was run with all 4 cytokines at 0 pg/mL, with and without 10 pg/mL eotaxin and 0.1 μg/mL of its biotinylated detection antibody to assess the effect on backgrounds. For each of the proteins, the backgrounds increased between 2.3-6.1-fold upon addition of eotaxin, an increase not anticipated by the 20% increase in detection antibody concentration. Significant cross-reactivity with eotaxin reagents may be inferred giving rise to false positive signals, so eotaxin was not added to this multiplex assay.

TABLE A

| | | AEB from IL-1b beads |
|---|---|---|
| | AEB from | IL-1b reagents plus 100 pg/mL |
| | IL-1b beads | of 3 cytokines and |
| [IL-1b] (pg/mL) | IL-1b reagents only | their detection antibodies |
| 0 | 0.0036 ± 0.0004 | 0.0118 ± 0.0007 |
| 1 | 0.2525 ± 0.0047 | 0.2355 ± 0.0319 |
| 10 | 2.159 ± 0.3658 | 2.229 ± 0.3973 |
| 100 | 15.86 ± 1.295 | 16.30 ± 2.243 |

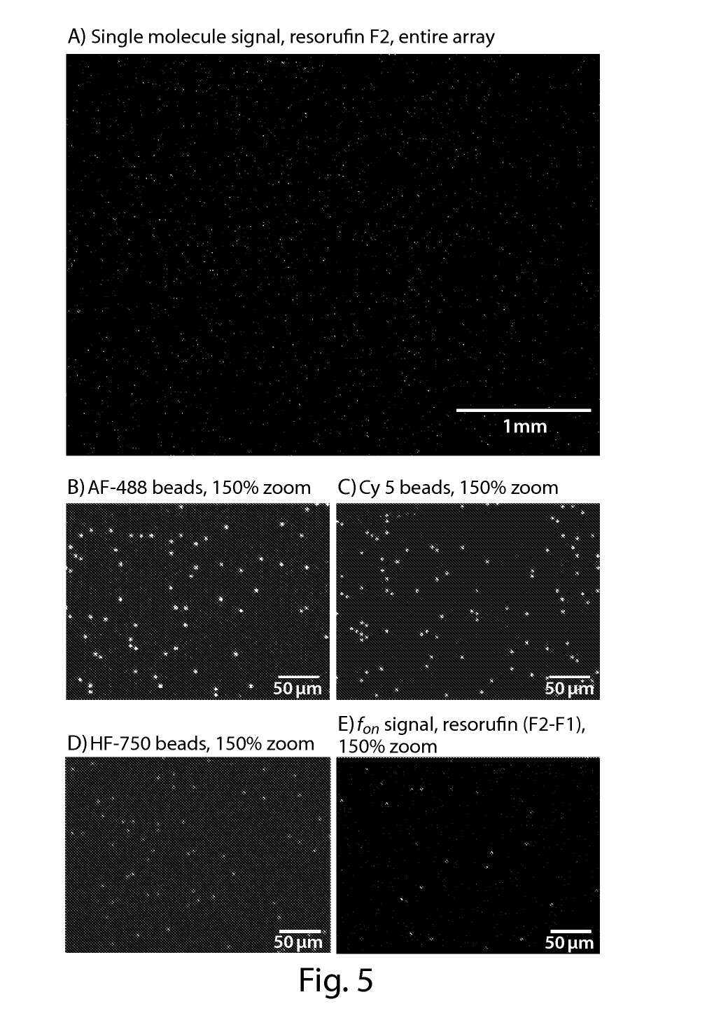

After minimizing the occurrence of false positives, a multiplex digital ELISA based on the approach in FIG. 3 for simultaneously measuring the concentrations of 4 cytokines (TNF-α, IL-6, IL-1α, and IL-1β) in plasma was developed. Details of the preparation of reagents, the assay steps used to form immunocomplexes, Simoa imaging, and image analysis used to decode each bead and to determine AEB values for the 4 cytokines are provided in the Methods. FIG. 5 shows representative images of the different wavelength imaged.

In FIG. 5: Representative images of an array from multiplexed digital ELISA at: A) & E) 574/615 nm ex/em; B) 490/530 nm ex/em; C) 622/667 nm ex/em; D) 740/800 nm ex/em.

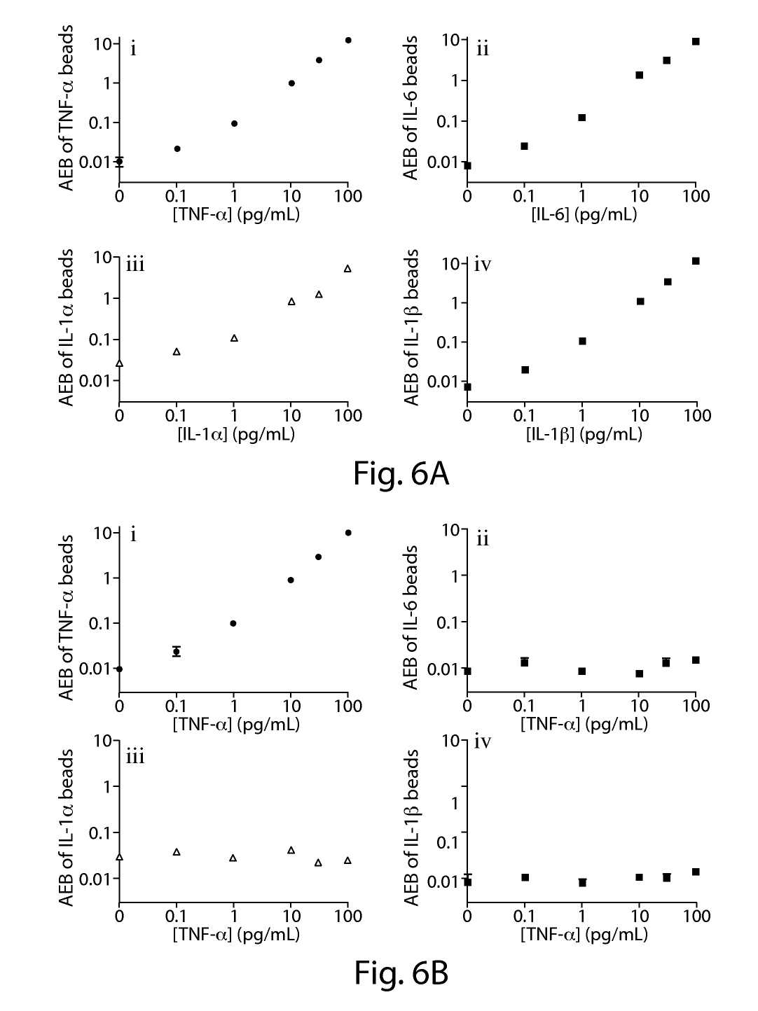

To evaluate the sensitivity and specificity of this 4-plex digital ELISA, AEB values were determined for samples in which: a) all four proteins were spiked into bovine serum (our calibration matrix) from femtomolar up to picomolar concentrations; and b) each individual protein was spiked into bovine serum separately. The first samples indicate the ability to measure 4 proteins simultaneously at femtomolar concentrations (sensitivity); the second set of samples would indicate the occurrence of false positives in the 3 non-spiked proteins (specificity). FIG. 6 show plots of AEB against concentrations of 4 cytokines from these samples; Table 3 provides the AEB values for each sample. The limits of detection (LODs) determined by interpolating the concentration at 3 s.d. of the background above background were 21 & 69 fg/mL (1.2 & 3.9 fM), 3 & 24 fg/mL (0.15 & 1.2 fM), 5 & 27 fg/mL (0.3 & 1.5 fM), and 43 & 32 fg/mL (2.5 & 1.9 fM), for TNF-α, IL-6, IL-1α, and IL-1β, respectively, in these two spiking experiments. These LODs are comparable to our previously reported values for non-encoded, single-plex digital ELISAs for TNF-α (11 fg/mL) and IL-6 (10 fg/mL), and encoded, single-plex digital ELISAs for all 4 cytokines given differences in the CV of backgrounds for particular experiments (Table 4). No significant increases in backgrounds from false positive were observed in the 3 subpopulations of beads that did not have protein spiked into the sample, up to 10 pg/mL of the spiked protein. At 100 pg/mL spiked proteins, most of the backgrounds were not elevated, although slight increases in signals from TNF-α beads spiked with 100 pg/mL of IL-1α and IL-1β (Table 3) were observed. These data indicate that multiplexed digital ELISA can provide similar sensitivity, specificity, and dynamic range as the single-plex approach.

TABLE 3
AEB as a function of concentration for calibration curves for example, as shown in FIG. 6.

| | TNF- α beads | | | | IL-6 beads | | | | IL-1 α beads | | | | IL-1 β beads | | | |
|---|---|---|---|---|---|---|---|---|---|---|---|---|---|---|---|---|
| | [cyto- | | | | [cyto- | | | | [cyto- | | | | [cyto- | | | |
| | kine] | | | | kine] | | | | kine] | | | | kine] | | | |
| Exper- | pg/ | | | CV | pg/ | | | CV | pg/ | | | CV | pg/ | | | CV |
| iment | mL | AEB | s.d. | (%) | mL | AEB | s.d. | (%) | mL | AEB | s.d. | (%) | mL | AEB | s.d. | (%) |
| TNF-α | 0 | 0.0091 | 0.0011 | 12% | 0 | 0.0086 | 0.0016 | 19% | 0 | 0.0306 | 0.0029 | 10% | 0 | 0.0083 | 0.0038 | 45% |
| only | 0.1 | 0.0246 | 0.0059 | 24% | 0.1 | 0.0127 | 0.0041 | 32% | 0.1 | 0.0377 | 0.0057 | 15% | 0.1 | 0.0106 | 0.0009 | 9% |
| spiked | 1 | 0.0972 | 0.0079 | 8% | 1 | 0.0086 | 0.0005 | 6% | 1 | 0.0283 | 0.0028 | 10% | 1 | 0.0081 | 0.0016 | 19% |
| in | 10 | 0.9197 | 0.0328 | 4% | 10 | 0.0074 | 0.0013 | 18% | 10 | 0.0411 | 0.0034 | 8% | 10 | 0.0107 | 0.0015 | 14% |
| | 30 | 3.0050 | 0.0799 | 3% | 30 | 0.0127 | 0.0032 | 25% | 30 | 0.0233 | 0.0035 | 15% | 30 | 0.0102 | 0.0022 | 22% |
| | 100 | 10.3392 | 0.4893 | 5% | 100 | 0.0151 | 0.0013 | 9% | 100 | 0.0259 | 0.0014 | 5% | 100 | 0.0142 | 0.0023 | 16% |
| IL-6 | 0 | 0.0068 | 0.0008 | 12% | 0 | 0.0108 | 0.0001 | 1% | 0 | 0.0271 | 0.0058 | 22% | 0 | 0.0090 | 0.0008 | 9% |
| only | 0.1 | 0.0115 | 0.0034 | 30% | 0.1 | 0.0245 | 0.0012 | 5% | 0.1 | 0.0321 | 0.0018 | 6% | 0.1 | 0.0102 | 0.0034 | 34% |
| spiked | 1 | 0.0072 | 0.0016 | 23% | 1 | 0.1218 | 0.0071 | 6% | 1 | 0.0251 | 0.0018 | 7% | 1 | 0.0114 | 0.0010 | 8% |
| in | 10 | 0.0110 | 0.0017 | 15% | 10 | 1.1289 | 0.0415 | 4% | 10 | 0.0309 | 0.0036 | 12% | 10 | 0.0089 | 0.0007 | 8% |
| | 30 | 0.0166 | 0.0026 | 16% | 30 | 3.8783 | 0.3436 | 9% | 30 | 0.0253 | 0.0034 | 13% | 30 | 0.0109 | 0.0018 | 16% |
| | 100 | 0.0254 | 0.0031 | 12% | 100 | 11.895 | 0.4263 | 4% | 100 | 0.0366 | 0.0044 | 12% | 100 | 0.0224 | 0.0019 | 8% |
| IL-1α | 0 | 0.0062 | 0.0001 | 1% | 0 | 0.0067 | 0.0013 | 19% | 0 | 0.0195 | 0.0004 | 2% | 0 | 0.0093 | 0.0021 | 23% |
| only | 0.1 | 0.0063 | 0.0021 | 34% | 0.1 | 0.0077 | 0.0004 | 6% | 0.1 | 0.0445 | 0.0045 | 10% | 0.1 | 0.0064 | 0.0009 | 14% |
| spiked | 1 | 0.0071 | 0.0009 | 13% | 1 | 0.0062 | 0.0011 | 18% | 1 | 0.0975 | 0.0052 | 5% | 1 | 0.0067 | 0.0005 | 8% |
| in | 10 | 0.0091 | 0.0016 | 17% | 10 | 0.0067 | 0.0011 | 17% | 10 | 0.8641 | 0.0119 | 1% | 10 | 0.0091 | 0.0018 | 19% |
| | 30 | 0.0255 | 0.0026 | 10% | 30 | 0.0126 | 0.0017 | 14% | 30 | 1.2379 | 0.0220 | 2% | 30 | 0.0098 | 0.0018 | 18% |
| | 100 | 0.0371 | 0.0003 | 1% | 100 | 0.0158 | 0.0008 | 5% | 100 | 3.9964 | 0.2728 | 7% | 100 | 0.0130 | 0.0021 | 16% |
| IL-1β | 0 | 0.0058 | 0.0010 | 16% | 0 | 0.0075 | 0.0018 | 24% | 0 | 0.0221 | 0.0021 | 9% | 0 | 0.0075 | 0.0014 | 19% |
| only | 0.1 | 0.0072 | 0.0015 | 21% | 0.1 | 0.0058 | 0.0006 | 11% | 0.1 | 0.0337 | 0.0068 | 20% | 0.1 | 0.0173 | 0.0043 | 25% |
| spiked | 1 | 0.0064 | 0.0014 | 22% | 1 | 0.0070 | 0.0026 | 37% | 1 | 0.0233 | 0.0060 | 26% | 1 | 0.0969 | 0.0128 | 13% |
| in | 10 | 0.0101 | 0.0005 | 5% | 10 | 0.0074 | 0.0021 | 29% | 10 | 0.0269 | 0.0056 | 21% | 10 | 1.0688 | 0.0463 | 4% |
| | 30 | 0.0163 | 0.0040 | 25% | 30 | 0.0152 | 0.0021 | 14% | 30 | 0.0235 | 0.0034 | 14% | 30 | 3.3097 | 0.3495 | 11% |
| | 100 | 0.0302 | 0.0033 | 11% | 100 | 0.0228 | 0.0040 | 17% | 100 | 0.0307 | 0.0030 | 10% | 100 | 12.6250 | 1.5968 | 13% |
| All 4 | 0 | 0.0100 | 0.0027 | 27% | 0 | 0.0078 | 0.0013 | 16% | 0 | 0.0268 | 0.0022 | 8% | 0 | 0.0074 | 0.0014 | 19% |
| cyto- | 0.1 | 0.0218 | 0.0022 | 10% | 0.1 | 0.0240 | 0.0026 | 11% | 0.1 | 0.0515 | 0.0056 | 11% | 0.1 | 0.0207 | 0.0031 | 15% |
| kines | 1 | 0.0949 | 0.0074 | 8% | 1 | 0.1248 | 0.0029 | 2% | 1 | 0.1085 | 0.0126 | 12% | 1 | 0.1045 | 0.0127 | 12% |
| spiked | 10 | 1.0169 | 0.0112 | 1% | 10 | 1.3811 | 0.0416 | 3% | 10 | 0.8517 | 0.0502 | 6% | 10 | 1.0982 | 0.0146 | 1% |
| in | 30 | 3.9060 | 0.3309 | 8% | 30 | 3.1087 | 0.2959 | 10% | 30 | 1.2566 | 0.0363 | 3% | 30 | 3.4399 | 0.2560 | 7% |
| | 100 | 12.3860 | 0.5393 | 4% | 100 | 9.0958 | 0.4408 | 5% | 100 | 5.2287 | 0.3311 | 6% | 100 | 12.4415 | 0.3068 | 2% |

TABLE 4
Limits of detection of 4 cytokines measured in multiplex and single-plex digital
ELISA. The CV of the background is given in each case, as that is an important
parameter for determining LOD.

| | LOD | LOD | CV of | |
|---|---|---|---|---|
| Cytokine | (fg/mL) | (fM) | background* | Source |
| TNF-α | 69 | 3.9 | 27% | Multiplex; this work; all 4 cytokines spiked in |
| | 21 | 1.2 | 12% | Multiplex; this work; only TNF-α spiked in |
| | 11 | 0.6 | 6% | Single-plex; this work, encoded beads |
| | 11 | 0.6 | 12% | Single-plex; Song et al.,** non-encoded beads |
| IL-6 | 24 | 1.2 | 16% | Multiplex; this work; all 4 cytokines spiked in |
| | 3 | 0.15 | 1% | Multiplex; this work; only IL-6 spiked in |
| | 4 | 0.2 | 9% | Single-plex; this work, encoded beads |
| | 10 | 0.5 | 8% | Single-plex; Song et al.,** non-encoded beads |
| IL-1α | 27 | 1.5 | 8% | Multiplex; this work; all 4 cytokines spiked in |
| | 5 | 0.3 | 2% | Multiplex; this work; only IL-1α spiked in |
| | 24 | 1.3 | 12% | Single-plex, this work, encoded beads |
| IL-1β | 32 | 1.9 | 19% | Multiplex; this work; all 4 cytokines spiked in |
| | 43 | 2.5 | 19% | Multiplex; this work; only IL-1β spiked in |
| | 12 | 0.7 | 10% | Single-plex, this work, encoded beads |

*LODs were determined using a 3 s.d. method, including those calculated from data in Song et al.**.
**see Song, L.; Hanlon, D. W.; Chang, L.; Provuncher, G. K.; Kan, C. W.; Campbell, T. G.; Fournier, D. R.; Ferrell, E. P.; Rivnak, A. J.; Pink, B. A.; Minnehan, K. A.; Patel, P. P.; Wilson, D. H.; Till M. A.; Faubion, W. A.; Duffy, D. C. Single molecule measurements of tumor necrosis factor α and interleukin-6 in the plasma of patients with Crohn's disease. J. Immunol. Methods 2011, 372, 177-86., herein incorporated by reference.

In FIG. 6: Plots of AEB against protein concentration for 4 beads specific to 4 cytokines measured in bovine serum samples spiked with: A) all 4 cytokines (i: AEB of TNF-α bead; ii: AEB of IL-6 beads; iii: AEB of IL-1α beads; iv: AEB of IL-1β beads); and B) only TNF-α (i: AEB of TNF-α bead; ii: AEB of IL-6 beads; iii: AEB of IL-1α beads; iv: AEB of IL-1β beads).

This assay to simultaneously measure the concentrations of the 4 cytokines in plasma from 15 healthy humans (Table 5). The concentrations of TNF-α, IL-6, IL-1α, and IL-1β were in the range (mean±s.d) 3.8-8.5 (5.4±1.2), 1.4-16.0 (4.1±3.6), 0.33-1.62 (0.87±0.41), and 0.65-12.1 (4.8±3.5) pg/mL, respectively. All cytokines were detected in all samples, except two samples in which IL-1α was not detected. The mean concentration of IL-6 was close to that previously measured in plasma using single-plex digital ELISA (3 pg/mL); the concentration of TNF-α, was higher than previously (3 pg/mL), which may be due to differences in collection method of plasma. All 4 cytokines were in the low- or sub-pg/mL range. Analog multiplex immunoassays typically have LOD greater than 5 pg/mL, so many of the cytokines would have been undetected or, for those that would have been detected, the imprecision would have been high.

TABLE 5
Concentrations of 4 cytokines measured in the plasma of 15 healthy
human donors using multiplex digital ELISA. Concentrations are given
as the mean and standard deviation of three replicates.

| Sample | [TNF-α] | [IL-6] | [IL-1α] | [IL-1β] |
|---|---|---|---|---|
| ID | (pg/mL) | (pg/mL) | (pg/mL) | (pg/mL) |
| 1 | 8.45 ± 1.11 | 5.02 ± 0.61 | 0.96 ± 0.37 | 8.84 ± 1.02 |
| 2 | 5.32 ± 0.39 | 4.99 ± 0.45 | 0.91 ± 0.21 | 1.51 ± 0.13 |
| 3 | 5.32 ± 1.56 | 3.68 ± 0.88 | 0.67 ± 0.11 | 2.99 ± 0.58 |
| 4 | 6.16 ± 1.51 | 1.68 ± 0.27 | 0.33 ± 0.14 | 2.82 ± 0.56 |
| 5 | 5.73 ± 1.18 | 4.48 ± 0.30 | 0.33 ± 0.24 | 6.51 ± 1.32 |
| 6 | 6.95 ± 1.89 | 4.89 ± 1.06 | 0.62 ± 0.36 | 1.96 ± 0.43 |
| 7 | 5.89 ± 1.36 | 2.92 ± 0.68 | not detected | 6.32 ± 1.49 |
| 8 | 3.79 ± 0.41 | 1.74 ± 0.31 | 0.52 ± 0.23 | 1.06 ± 0.17 |
| 9 | 4.43 ± 0.64 | 16.0 ± 3.4 | not detected | 0.65 ± 0.06 |
| 10 | 3.78 ± 0.96 | 1.56 ± 0.36 | 1.50 ± 0.38 | 8.09 ± 2.04 |
| 11 | 4.07 ± 0.82 | 1.37 ± 0.13 | 1.16 ± 0.34 | 4.55 ± 0.94 |
| 12 | 5.85 ± 0.20 | 3.61 ± 0.41 | 1.62 ± 0.26 | 12.1 ± 0.8 |
| 13 | 4.73 ± 0.24 | 2.66 ± 0.37 | 0.84 ± 0.36 | 2.41 ± 0.13 |
| 14 | 5.43 ± 0.51 | 4.97 ± 0.59 | 1.17 ± 0.40 | 2.79 ± 0.34 |
| 15 | 4.92 ± 0.46 | 2.02 ± 0.16 | 0.67 ± 0.20 | 8.86 ± 0.49 |

This work has provided a demonstration of multiplexing 4 proteins using this method. Multiple proteins can be measured simultaneously at the single molecule level using Simoa. The ability to reliably detect and quantify low concentrations of multiple proteins in clinical samples could have a major impact on the ability to assess the status of complex pathways in biological samples in one experiment.

### Example 2

This example described a non-limiting method for determining the average number of dye molecules associated with each of a plurality of beads.

First, unactivated beads were mixed with solutions of a variety of known concentrations of three different dyes (see Tables 6A-C). A bead solution was injected onto a Simoa disc (see Example 1) and the reaction vessels were sealed with oil (see Example 1). Calibration curves of dye concentration versus average signal from individual wells were prepared for each dye.

Next, the dyes were coupled to activated beads using labeling method described in Example 1. The beads were injected in a Simoa disc and sealed with oil, using the same method as used for the preparing the calibration curve. The average signal of beads in wells were determined. The concentration of dye on the bead was interpreted from the calibration curves (see Tables 7A-C).

TABLE 6A
First calibration curve.

| 488 nm | 488 nm | | | | |
|---|---|---|---|---|---|
| Curve | Average | — | — | — | Molecules |
| [μM] | Intensity | sd | cv % | Dye, [M] | Dye/Well |
| 0 | 128.0 | 11.0 | 9% | 0.00E+00 | 0 |
| 2.5 | 651.4 | 36.8 | 6% | 2.50E−06 | 48629 |
| 5 | 1387.7 | 100.8 | 7% | 5.00E−06 | 97259 |
| 10 | 2590.0 | 145.7 | 6% | 1.00E−05 | 194518 |

TABLE 6B
Second calibration curve.

| | 647 nm | | | | |
|---|---|---|---|---|---|
| 647 nm Dye | Average | — | — | — | Molecules |
| [μM] | Intensity | sd | cv % | Dye, [M] | Dye/Well |
| 0 | 75.7 | 26.2 | 35% | 0.00E+00 | 0 |
| 1 | 681.6 | 46.4 | 7% | 1.00E−06 | 19452 |
| 2.5 | 1632.9 | 71.5 | 4% | 2.50E−06 | 48629 |
| 5 | 3205.5 | 169.4 | 5% | 5.00E−06 | 97259 |

TABLE 6C
Third calibration curve.

| 750 nm | 750 nm | | | | |
|---|---|---|---|---|---|
| Curve | Average | — | — | Dye | Molecules |
| [AM] | Intensity | sd | cv % | [M] | Dye/Well |
| 0 | 25.7 | 5.6 | 22% | 0.00E+00 | 0 |
| 1 | 175.3 | 8.3 | 5% | 1.00E−06 | 19452 |
| 2.5 | 425.0 | 13.6 | 3% | 2.50E−06 | 48629 |
| 5 | 735.0 | 71.2 | 10% | 5.00E−06 | 97259 |

TABLE 7A

| 488 | Calculated | | | | Dye, | Molecules/ |
|---|---|---|---|---|---|---|
| bead | [μM] | Avg | sd | cv % | [M] | Bead |
| “16.5” | 6.78 | 1789.8 | 267.2 | 6% | 6.78E−06 | 131,888 |

TABLE 7B

| 647 | Calculated | | | | Dye, | Molecules/ |
|---|---|---|---|---|---|---|
| beads | [μM] | Avg | sd | cv % | [M] | Bead |
| “8” (low) | 0.02 | 76.4 | 8.9 | 12% | 1.69E−08 | 328 |
| “12” (high) | 0.83 | 586.7 | 99.6 | 17% | 8.30E−07 | 16,150 |

TABLE 7C

| 750 nm | Calculated | | | | Dye, | Molecules/ |
|---|---|---|---|---|---|---|
| bead | [μM] | Avg | sd | cv % | [M] | Bead |
| “17” | 1.24 | 214.4 | 31.1 | 15% | 1.24E−06 | 24,142 |

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

The indefinite articles “a” and “an,” as used herein in the specification and in the claims, unless clearly indicated to the contrary, should be understood to mean “at least one.”

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively.

What is claimed:

1. A method for covalently associating a molecular species with a surface, comprising:
exposing an object with a surface comprising a plurality of functional groups to a first type of molecular species, wherein at least some of the plurality of functional groups each covalently associate with the first type of molecular species and at least some of the plurality of functional groups do not associate with any of the first type of molecular species;
deactivating the functional groups not associated with the first type of molecular species to form a plurality of deactivated functional groups;
reactivating the plurality of deactivated functional groups to form a plurality of reactivated functional groups; and
exposing the objects to a second type of molecular species, wherein at least some of the plurality of reactivated functional groups each covalently associate with a second type of molecular species.

2. The method of any preceding claim, wherein the plurality of the first type of molecular species comprising a plurality of reporter molecules

3. The method of claim 2, wherein the reporter molecule is a dye.

4. The method of claim 3, wherein the dye is a hydrazide dye.

5. The method of any preceding claim, wherein the plurality of a second type of molecular species comprises a plurality of targeting entities.

6. The method of claim 5, wherein the targeting entity is a protein.

7. The method of any preceding claim, wherein the plurality of functional groups comprises a plurality of carboxylic acids, amides, or thiols.

8. The method of any preceding claim, wherein the plurality of functional groups comprises a plurality of carboxylic acid groups associated with a chemical crosslinker.

9. The method of claim 8, wherein the plurality of carboxylic acid groups associated with a chemical crosslinker are deactivated by hydrolyzation, wherein the deactivated groups comprise a carboxylic acid.

10. The method of claim 9, wherein the deactivated groups comprising carboxylic acid are reactivated by exposure and reaction with the chemical crosslinker.

11. The method of any preceding claim, wherein the deactivating comprises exposing the object to a deactivating agent, wherein the deactivating agent associates or reacts with the functional group to form a deactivated group.

12. The method of claim 11, wherein the functional group is a thiol and the deactivated group comprises a disulfide.

13. The method of claim 12, wherein the disulfide is reactivated by exposure to a disulfide reducing agent.

14. The method of any preceding claim, wherein the functional group comprises a photoreactive group.

15. The method of claim 14, wherein the functional group comprising a photoreactive group is deactivated by removal of UV light, thereby forming a deactivated photoreactive group.

16. The method of claim 15, wherein the deactivated photoreactive group is reactivated by exposure to UV light.

17. The method of any preceding claim, wherein a method comprises a plurality of objects.

18. The method of claim 17, wherein the plurality of objects comprises a plurality of beads.

19. The method of any preceding claim, wherein the object comprises a bead.

20. The method of any preceding claim, wherein the average diameter of the plurality of objects is between about 0.1 micrometer and about 100 micrometers.

21. The method of any preceding claim, wherein the average diameter of the plurality of objects is between about 1 micrometer and about 10 micrometers.

22. The method of any preceding claim, further comprising performing at least one wash step.

23. An activated material capable of being covalently functionalized with a first type of molecular species, comprising:
a plurality of functional groups associated with at least a portion of the surface of the activated material, wherein
at least a portion of the functional groups are associated with the first type of molecular species; and
at least a portion of the functional groups are not associated with the first type of molecular species but are instead deactivated and capable of being reactivation and of becoming covalently associated with a second type of molecular species.

24. The material of claim 23, wherein the activated material comprises a plurality of beads.

25. A kit, comprising:
a plurality of materials as in claim 23 or 24, wherein each material is distinguishable from each and every other material.
